(19) United States
(12) Patent Application Publication
(10) Pub. No.: US 2017/0065424 A1
(43) Pub. Date: Mar. 9, 2017

(54) Small Bone Orthopedic Implants

(71) Applicant: Centric Medical, LLC, Huntley, IL (US)

(72) Inventors: Garrett D. Lauf, Elgin, IL (US); Michael S. Butler, St. Charles, IL (US); Richard M. Mueller, St. Charles, IL (US); Matthew S. Coyne, Naperville, IL (US)

(73) Assignee: Centric Medical, LLC, Huntley, IL (US)

(21) Appl. No.: 15/256,530

(22) Filed: Sep. 3, 2016

Related U.S. Application Data
(60) Provisional application No. 62/214,612, filed on Sep. 4, 2015.
(60) Provisional application No. 62/254,949, filed on Nov. 13, 2015.
(60) Provisional application No. 62/314,455, filed on Mar. 29, 2016.

(51) Int. Cl. A61F 2/42

(52) U.S. Cl. CPC A61F 2/4225; A61F 2/4241; A61F 2002/4228; A61F 2002/4243; A61F 2002/30622

(57) ABSTRACT

Orthopedic implants described herein are used for fractures, inter-digital fusion of the fingers, toes, and other small bones of the body, as well as other procedures. Each implant has first and second drive ends for insertion into bone. The drive ends may be on the same or separate implant component(s) and include external threading. In one form, a single component includes the first and second drive ends, while in another form, a first component has the first drive end and a second component has the second drive end. In the two component version, a head of one component receives an end of the other component to provide coupling or joining of the two components.

### CROSS-REFERENCE TO RELATED APPLICATIONS

This U.S. non-provisional patent application claims the benefit of and/or priority under 35 U.S.C. §119(e) to U.S. provisional patent application Ser. No. 62/214,612 filed Sep. 4, 2015 titled “Hammertoe Implants,” U.S. provisional patent application Ser. No. 62/254,949 filed Nov. 13, 2015 titled “Small Bone Orthopedic Implants,” and U.S. provisional patent application Ser. No. 62/314,455 filed Mar. 29, 2016, the entire contents of each of which is specifically incorporated herein by reference.

### FIELD OF THE INVENTION

The present invention relates to orthopedic implants for bones of the body and, particularly, to orthopedic implants for small bones of the body such as, but not limited to, the foot and hand.

### BACKGROUND

Small bones of the body such as those of the foot and hand are susceptible to injury, trauma, disease, deformity, and other issues. Some of these issues develop over time due to various factors. While many issues may affect small bones of the body, hammer toe, mallet toe and claw toe are fairly common foot bone deformities that can develop and/or get worse over time. A hammer toe is characterized by a contracture (bending) down at the middle joint of a toe of the foot. A mallet toe is characterized by a contracture (bending) down at the joint of a toe nearest to the tip or toenail of the toe. Claw toe is characterized by one or more toes bending up at the joint where the toes and foot meet, then bend down at the middle joints and at the joints nearest to the tip or toenail of the toes, causing the toes to curl down toward the floor.

Hammertoe, mallet toe, claw toe, as well as other small bone issues, may be treated with non-surgical methods, typically depending on the severity of the condition. Non-surgical treatment includes padding, changes in footwear, orthotic devices, injection therapy, medications, splinting/strapping, as well as physical therapy. Sometimes however, the small bone condition does not respond to non-surgical treatment or the condition worsens over time.

In those cases where surgical treatment is desired or necessary, there are few viable options. One option is to use/implant an orthopedic device. Of the current orthopedic devices for small bone issues most, if not all, are deficient in various respects. It is therefore desirable to have an orthopedic implant for small bones of the body particularly, but not limited to, small bones of the foot and hand.

The present invention solves the problems of the prior art.

### SUMMARY OF THE INVENTION

The present invention is an orthopedic implant for use in small bones of the foot and hand. The present small bone orthopedic implant may be used for fractures and inter-digital fusion of the fingers, toes, and other small bones of the body.

One form of the present small bone orthopedic implant comprises a two component version. Variations of the two component version of the present small bone orthopedic implant are also provided. Another form of the present small bone orthopedic implant comprises a one component version. Variations of the one component version of the present small bone orthopedic implant are also provided. All components of the present small bone orthopedic implants shown and/or described herein are fashioned from a known biocompatible implant material.

The two component small bone orthopedic implant comprises first and second (e.g. male and female) components or segments at least one segment, and mostly both segments, of which includes external threading. Each segment is designed to be driven separately into bone and thereafter connect to each other. The heads of the first (e.g. female) segment and of the male (e.g. second) segment include features that provide positive mating of the two segments when joined.

In one version, the longitudinal axis of the first segment and the longitudinal axis of the second segment are co-axial. In another version, the longitudinal axis of the first segment and the longitudinal axis of the second segment are skewed.

In one version, each segment is generally conical shaped and includes external threading extending from a head to a tip thereof. In another version, each segment is generally cylindrical shaped and includes external threading extending from a head to a tip thereof.

The one component small bone orthopedic implant comprises a rod having a trocar tip, a drive end opposite the trocar tip, first cortical threading proximate the trocar tip, and second cortical threading between the first cortical threading and the drive end. The first and second cortical threading having different pitches.

One variation of the one component small bone orthopedic implant includes a scored portion that allows the drive end of the rod to break away from the remainder of the rod.

Another version of the one component small bone orthopedic with the breakaway portion includes a shoulder between the first cortical threading and the second cortical threading.

A further version of the present small bone orthopedic implant is characterized by a dual-threaded screw having a shaft with first threading proximate a first end of the shaft, and second threading proximate a second end of the shaft, the first end having a pointed tip, and the second end having a drive socket configured to receive an implant driver, the pitch of the first threading being different than the pitch of the second threading.

In one form of this version, the second end of the shaft of the second component has a diameter that is greater than the first end of the shaft of the second component such that the diameter of the second threading is greater than the diameter of the first threading. The implant driver is characterized by a rod having a pointed tip at a first end and a drive head at a second end, the drive head configured for reception in the drive socket of the small bone orthopedic implant.

Further aspects of the present invention will become apparent from consideration of the drawings and the following description of forms of the invention. A person skilled in the art will realize that other forms of the invention are possible and that the details of the invention can be modified in a number of respects without departing from the inventive concept. The following drawings and description are to be regarded as illustrative in nature and not restrictive.

### BRIEF DESCRIPTION OF THE DRAWINGS

The features of the invention will be better understood by reference to the accompanying drawings which illustrate forms of the present invention, wherein:

FIG. 1 is an isometric view of an exemplary form of a two component small bone orthopedic implant fashioned in accordance with the present principles;

FIG. 2 is an exploded view of the two components of the small bone orthopedic implant of FIG. 1;

FIG. 3 is an isometric view of a first component of the two components of the small bone orthopedic implant of FIG. 1;

FIG. 4 is an isometric view of a second component of the two components of the small bone orthopedic implant of FIG. 1;

FIG. 5 is a side view of the small bone orthopedic implant of FIG. 1;

FIG. 6 is a sectional view of the small bone orthopedic implant of FIG. 5 taken along line 6-6 thereof;

FIG. 7 is a side view of an exemplary form of a variation of the small bone orthopedic implant of FIG. 1;

FIG. 8 is a sectional view of the small bone orthopedic implant of FIG. 7 taken along line 8-8 thereof;

FIG. 9 is a plan view of distal portions of the bones of a left foot with the small bone orthopedic implant of FIG. 1 implanted into a proximal and a middle phalange of the second toe of the left foot;

FIG. 10 is a plan view of distal portions of the bones of a left foot with the small bone orthopedic implant of FIG. 1 implanted into a proximal and a middle phalange of the second and fourth toes of the left foot and the small bone orthopedic implant of FIG. 7 implanted into a proximal and a middle phalange of the third toe of the left foot;

FIG. 11 is an isometric view of an exemplary form of a one piece small bone orthopedic implant fashioned in accordance with the present principles;

FIG. 12 is a side view of the one piece small bone orthopedic implant of FIG. 11;

FIG. 13 is another side view of the one piece small bone orthopedic implant of FIG. 11;

FIG. 14 is a sectional view of the one piece small bone orthopedic implant of FIG. 13 taken along line 14-14 thereof;

FIG. 15 is an isometric view of the one piece small bone orthopedic implant of FIG. 11;

FIG. 16 is an enlarged end view of the one piece small bone orthopedic implant of FIG. 11;

FIG. 17 is a plan view of distal portions of the bones of a left foot with the small bone orthopedic implant of FIG. 11 implanted into a proximal and a middle phalange of the second toe of the left foot;

FIG. 18 is a side view of another exemplary form of a one piece small bone orthopedic implant fashioned in accordance with the present principles;

FIG. 19 is an enlarged view of a breakaway section of the one piece small bone orthopedic implant of FIG. 18;

FIG. 20 is a side view of another exemplary form of a one piece small bone orthopedic implant fashioned in accordance with the present principles;

FIG. 21 is a sectional view of a portion of the one piece small bone orthopedic implant of FIG. 20 taken along line 21-21 thereof;

FIG. 22 is a sectional view of the one piece small bone orthopedic implant of FIG. 20 taken along line 22-22 thereof;

FIG. 23 is a side view of a portion of the one piece small bone orthopedic implant of FIG. 20;

FIG. 24 is an isometric exploded view showing another exemplary form of a small bone orthopedic implant and implant driver fashioned in accordance with the present principles;

FIG. 25 is an isometric view showing the implant driver engaged with the small bone orthopedic implant;

FIG. 26 is a sectional side view of the drive head of the implant driver being received in the drive socket of the small bone orthopedic implant;

FIG. 27 is a sectional side view of the drive head of the implant driver fully received in or engaged with the drive socket of the small bone orthopedic implant;

FIG. 28 is an enlarged sectional side view of a section of the distal end of the implant driver and the proximal end of the small bone orthopedic implant, showing the drive head of the implant driver fully received in or engaged with the drive socket of the small bone orthopedic implant;

FIG. 29 is another sectional side view of the drive head of the implant driver fully received in or engaged with the drive socket of the small bone orthopedic implant;

FIG. 30 is an enlarged isometric view of the drive head and a section of the distal end of the implant driver;

FIG. 31 is an isometric side view of the implant driver;

FIG. 32 is an enlarged sectional side view of the drive head and a section of the distal end of the implant driver;

FIG. 33 is a sectional side view of the implant driver;

FIG. 34 is a side view of the small bone orthopedic implant;

FIG. 35 is an isometric view of the small bone orthopedic implant;

FIG. 36 is a sectional side view of the small bone orthopedic implant;

FIG. 37 is an enlarged side view of a section of the distal end of the small bone orthopedic implant;

FIG. 38 is a side view of another exemplary form of a small bone orthopedic implant fashioned in accordance with the present principles;

FIG. 39 is another side view of the small bone orthopedic implant of FIG. 38;

FIG. 40 is an isometric view of the small bone orthopedic implant of FIG. 38;

FIG. 41 is a sectional view of the small bone orthopedic implant of FIG. 38 taken along line 41-41 thereof;

FIG. 42 is a sectional view of the small bone orthopedic implant of FIG. 39 taken along line 42-42 thereof;

FIG. 43 is a side view of the first component of the small bone orthopedic implant of FIG. 38;

FIG. 44 is another side view of the first component of the small bone orthopedic implant of FIG. 38;

FIG. 45 is a sectional view of the first component of the small bone orthopedic implant of FIG. 38 taken along line 45-45 of FIG. 43;

FIG. 46 is a sectional view of the first component of the small bone orthopedic implant of FIG. 38 taken along line 46-46 of FIG. 44;

FIG. 47 is an isometric view of the first component of the small bone orthopedic implant of FIG. 38;

FIG. 48 is a side view of the second component of the small bone orthopedic implant of FIG. 38;

FIG. 49 is another side view of the second component of the small bone orthopedic implant of FIG. 38;

FIG. 50 is a sectional view of the second component of the small bone orthopedic implant of FIG. 38 taken along line 50-50 of FIG. 48;

FIG. 51 is a sectional view of the second component of the small bone orthopedic implant of FIG. 38 taken along line 51-51 of FIG. 49; and

FIG. 52 is an isometric view of the second component of the small bone orthopedic implant of FIG. 38.

### DETAILED DESCRIPTION OF THE INVENTION

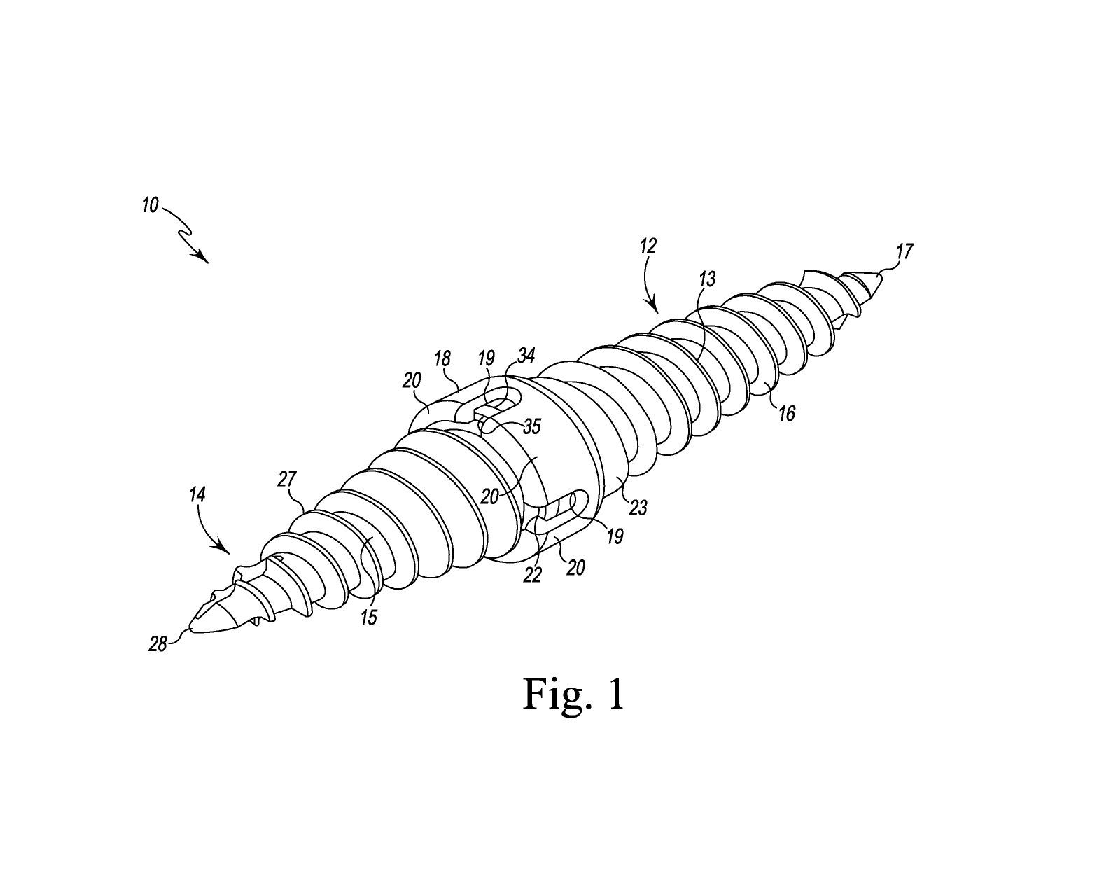

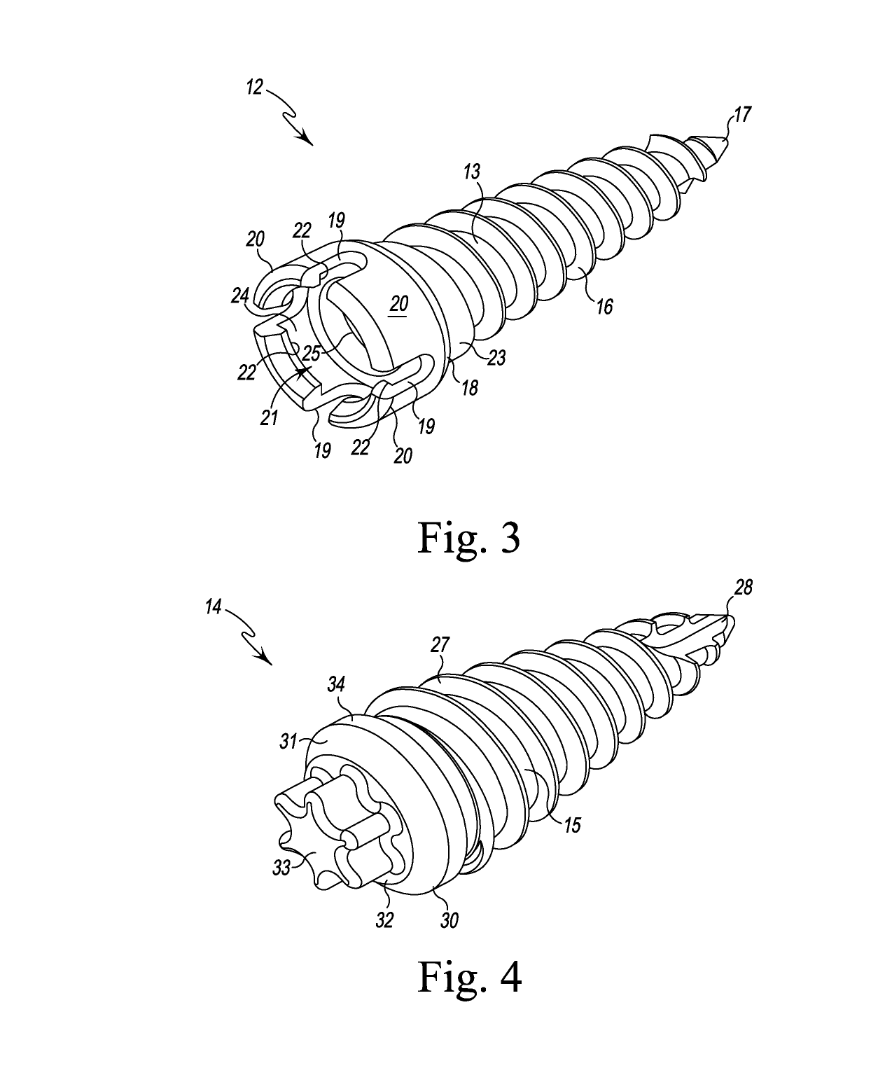

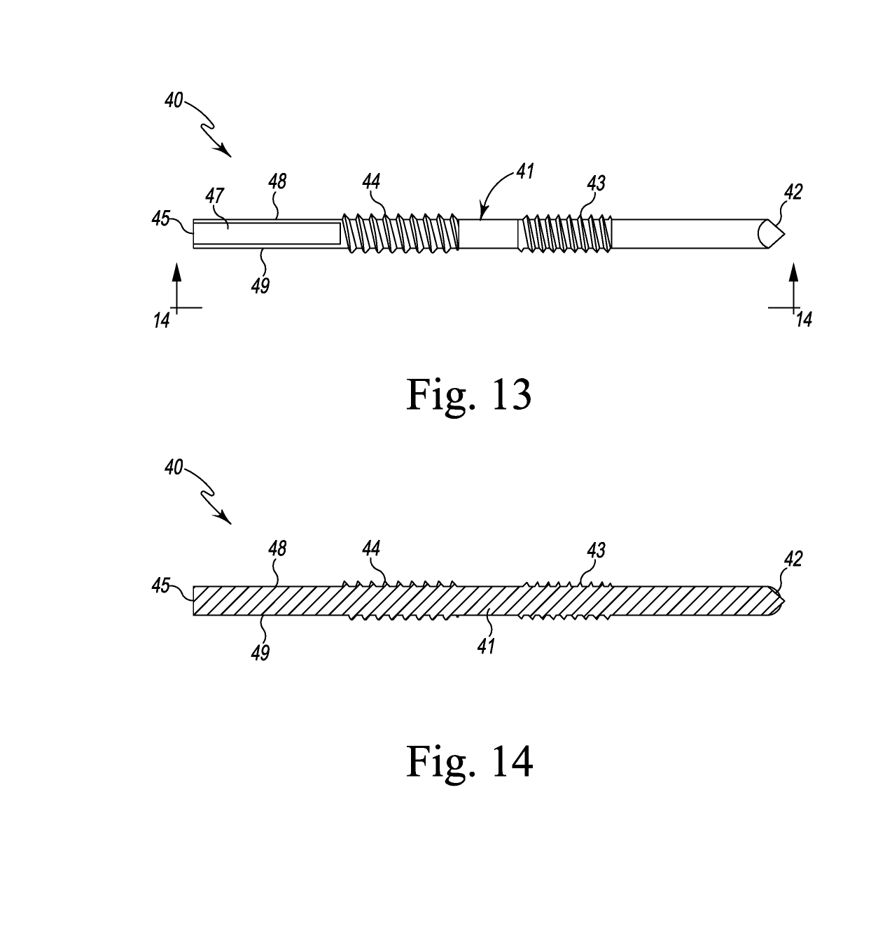

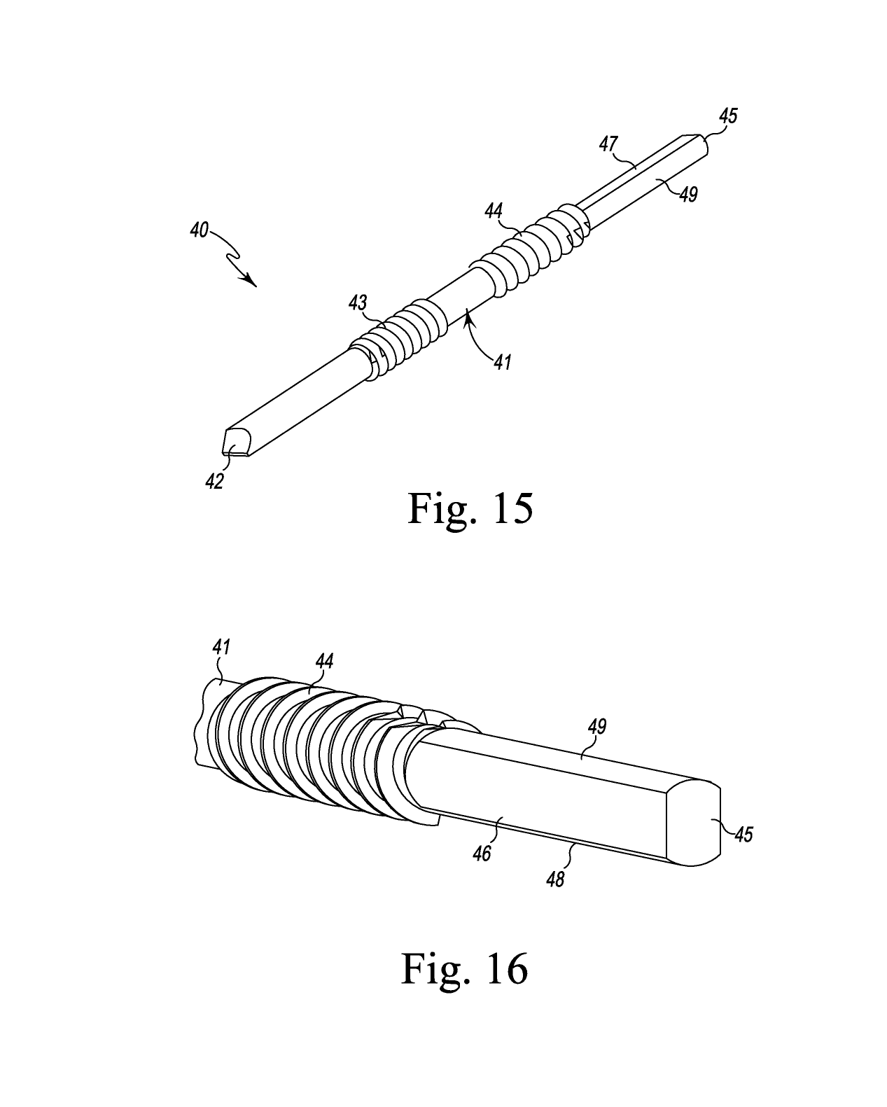

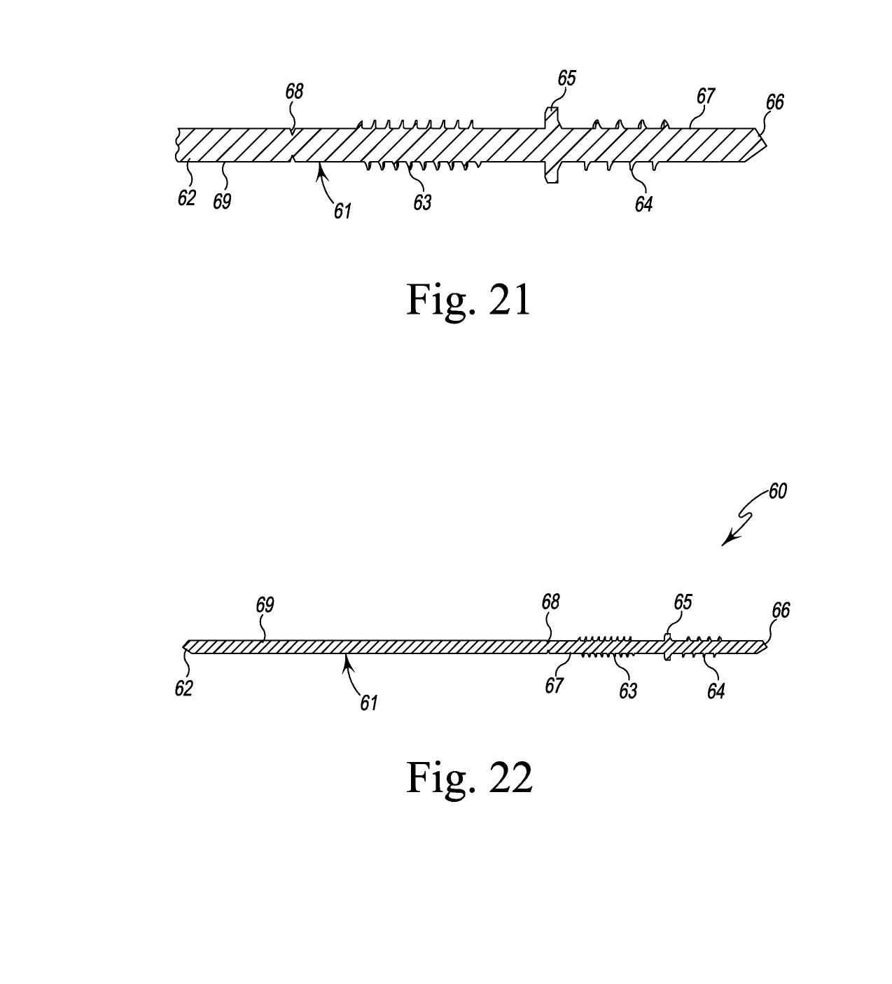

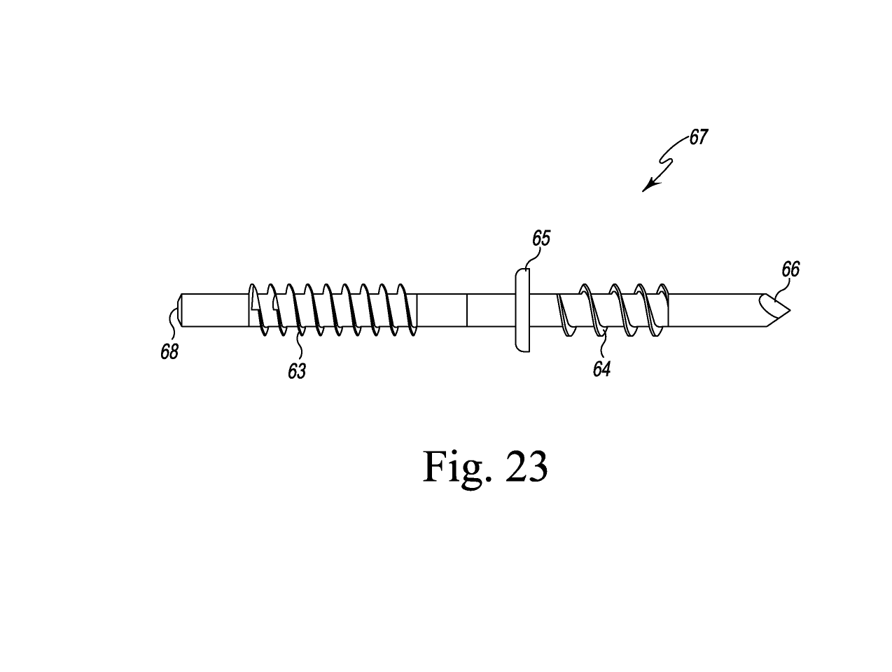

FIGS. 1-23 depict exemplary forms or versions of a small bone orthopedic implant fashioned in accordance with the present principles for treating ailments such as, but not limited to, deformities of small bones of the foot and the hand, as well as some of the exemplary forms of the present small bone orthopedic implant implanted into small bones of the foot. As with all of the present small bone orthopedic implants, they are to be used for fractures, inter-digital fusion of the fingers, toes, and other small bones, as well as other conditions. It should be appreciated, however, that the various forms of the present small bone orthopedic implant may be used with other bones of the body. The use described herein is only exemplary.

Referring to FIGS. 1-6, there is depicted a two component small bone orthopedic implant generally designated 10. The two component small bone orthopedic implant 10 is thus comprised of a first component or segment 12 and a second component or segment 14, the nomenclature first and second being arbitrary. The first and second components 12, 14 are fashioned from a biocompatible material such as, but not limited to, a titanium alloy and are configured to be joined to one another in the implant process. The first component 12 may be considered a female component while the second component 14 may be considered a male component.

The first component 12 is characterized by a generally cone shaped body 16 having a head 18 at one end thereof and a generally pointed tip 17 at another end thereof. The body 16 has distal cortical threads or threading 13 extending generally from the head 18 to the tip 17, the external threading 13 sized, shaped and pitched to provide adequate reception and retention in bone.

The head 18 is cup-shaped having a generally rounded walls 20 with a plurality of slots 19 formed in the walls 20. The walls 20 define a socket 21 having an end 25. The end 25 is configured in like manner as the boss 33 of the second component 14 (which is described more fully below). While the end 25 and thus the boss 33 is shown as generally hexagonal in shape, the end 25 and the boss 33 may be configured in other shapes as desired. The head 18 is configured to allow reception of a tool (not shown) to drive the first component 12 into a small bone of the body.

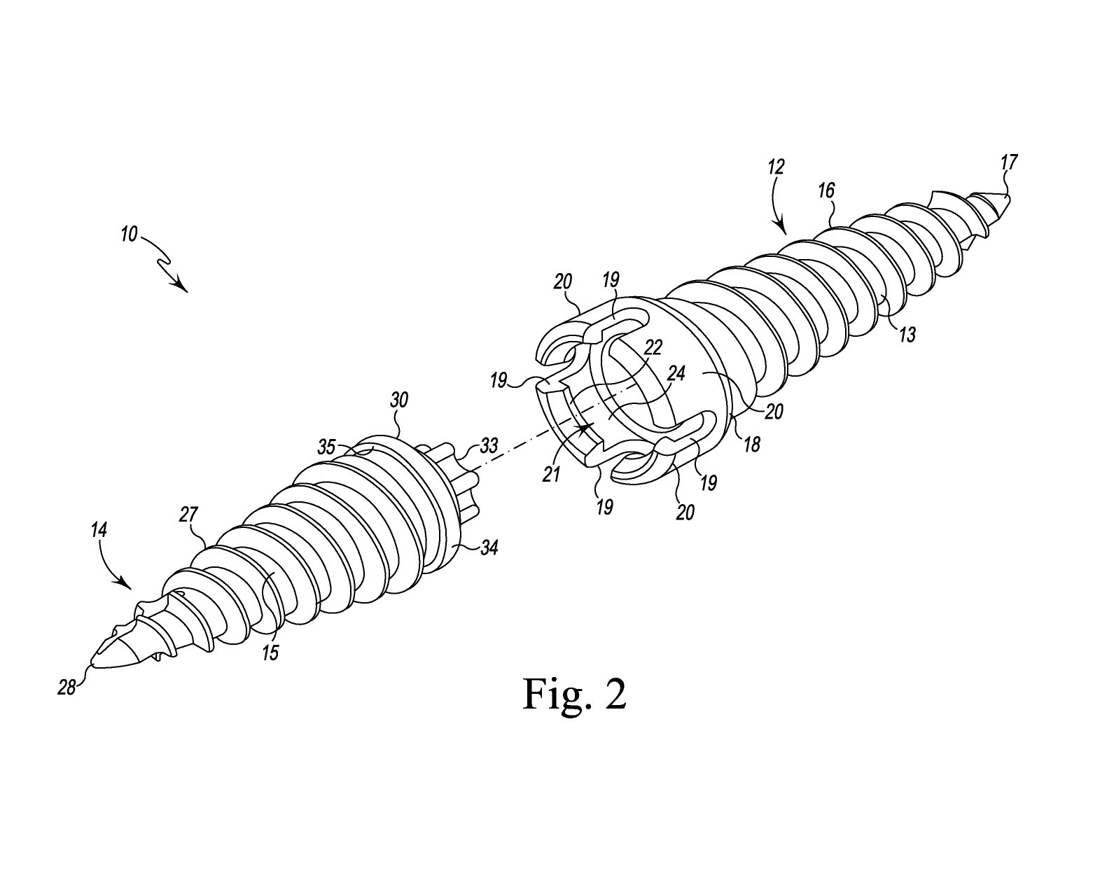

As best seen in FIGS. 2, 3, and 6, the inside of the walls 20 have an undercut or ledge 22 and an annular channel 24 axially below the ledges 22 that together define a generally annular retention tang providing a mating feature for coupling with the second component 14. The slots 19 in the walls 20 allow for the walls 20 to expand slightly when the second component 14 is inserted into the head 18 but resiliently return to their original shape after insertion of the second component 14.

The second component 14 is characterized by a generally cone shaped body 15 having a head 30 at one end thereof and a generally pointed tip 28 at another end thereof. The body 15 has distal cortical threads or threading 27 extending generally from the head 18 to the tip 28, the external threading 27 sized, shaped and pitched to provide adequate reception and retention in bone.

The head 30 is generally round in shape defining a round and flat middle 34 with a generally round and angled upper surface 31 terminating in a generally flat top 32. A boss or drive feature 33 is situated on and extends axially from the top 32. The boss 33 is configured for reception in the configured end 25 of the socket 21 of the first component. As such, the boss 33 is generally hexagonal in shape. The boss 33 may take other shapes as desired.

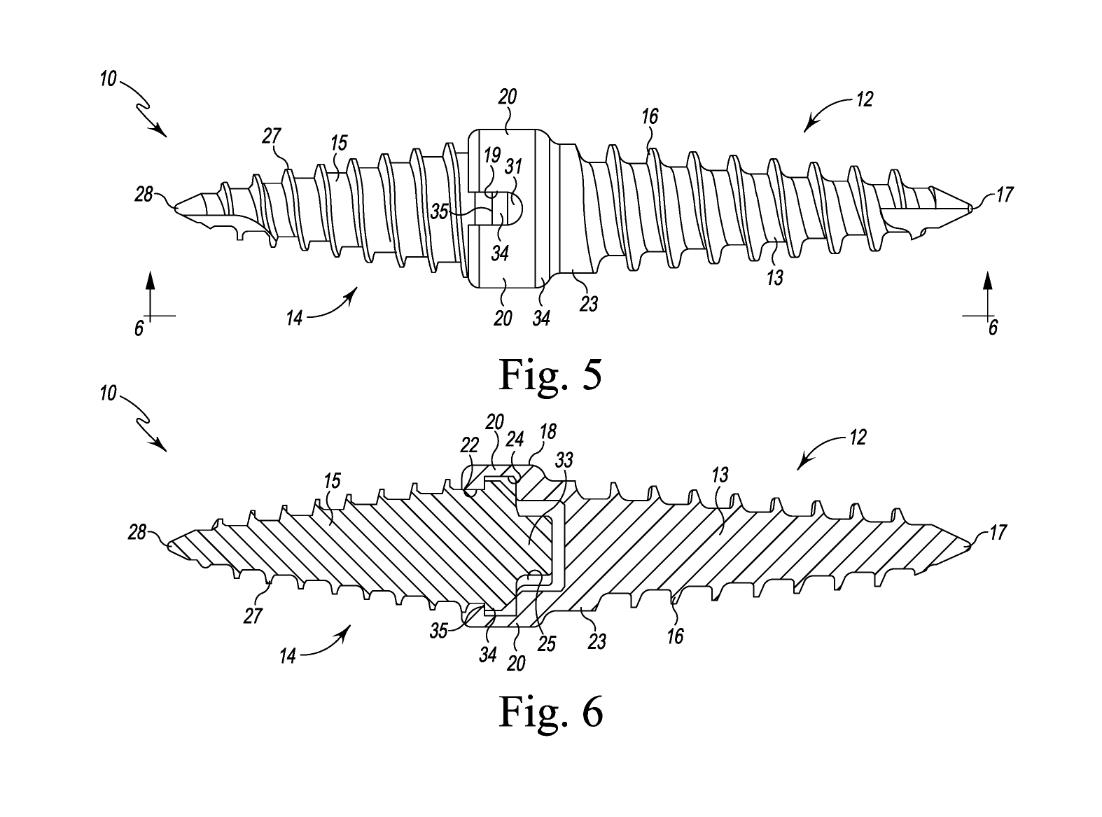

As best seen in FIGS. 4, 6, the head 30 of the second component 14 includes a proximal mating feature that retains the second (male) component 14 onto the first (female) component 12 preventing disassociation between the two components. In particular, the head 30 has an annular flat 35 axially under the round middle 34. The flat 34 abuts the ledges 22 of the first component 12 to prevent the second component 14 from dissociating from the first component 12 once joined.

The two component small bone orthopedic implant 10 is designed such that the longitudinal axis of the first component 12 and the longitudinal axis of the second component 14 are co-axial or straight relative to one another.

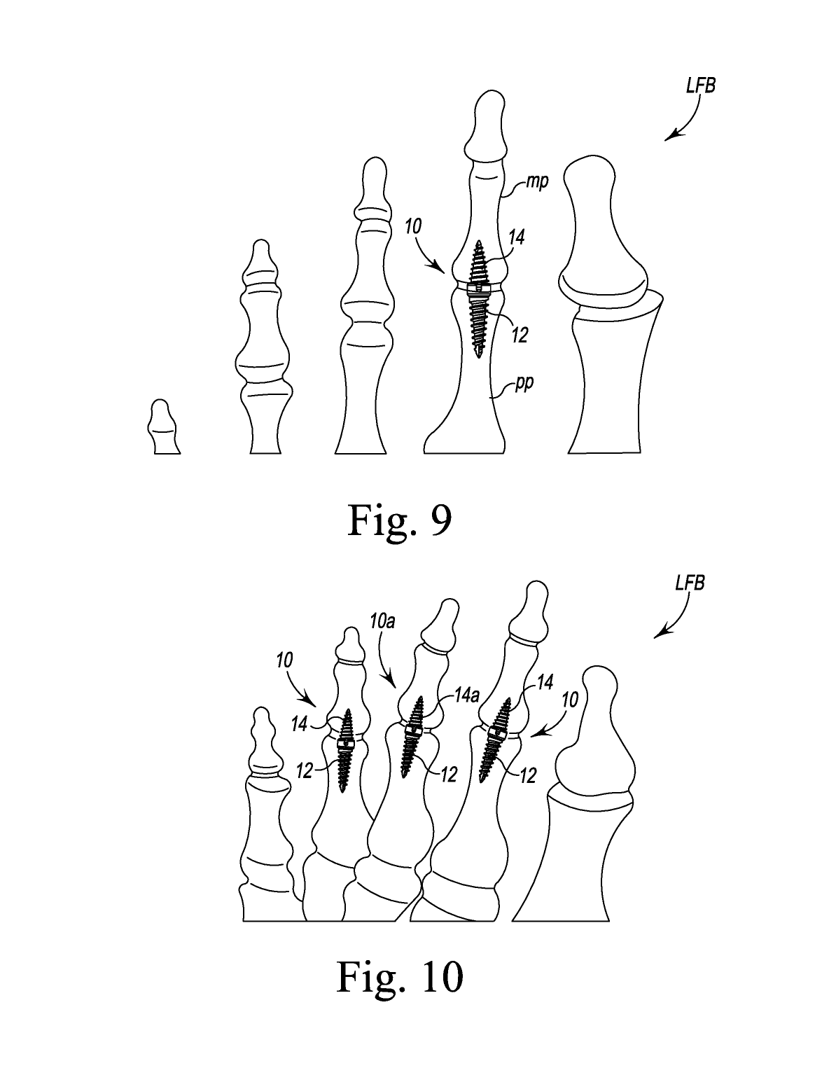

A manner of implanting the small bone orthopedic implant 10 (and all of the other small bone implants shown and/or described herein) for use in treating a hammer toe will now be presented. It should be appreciated that such use is only exemplary of one of many uses. The first or female component 12 will be placed first on the distal end of a proximal phalange of a toe of the foot. The top of the head 18 of the first component 12 is to be flush with the bone surface. The second or male component 14 is to then be inserted onto the proximal end of the middle phalange of the toe of a foot having the mating feature proud of the bone surface. The two components will then be pushed together keeping both concentric with each other until the retention feature of the first component captures the second component providing a stable construct to help promote fusion of the joint. In FIG. 9, the small bone orthopedic implant 10 is shown implanted in the second toe of a left foot (LFB).

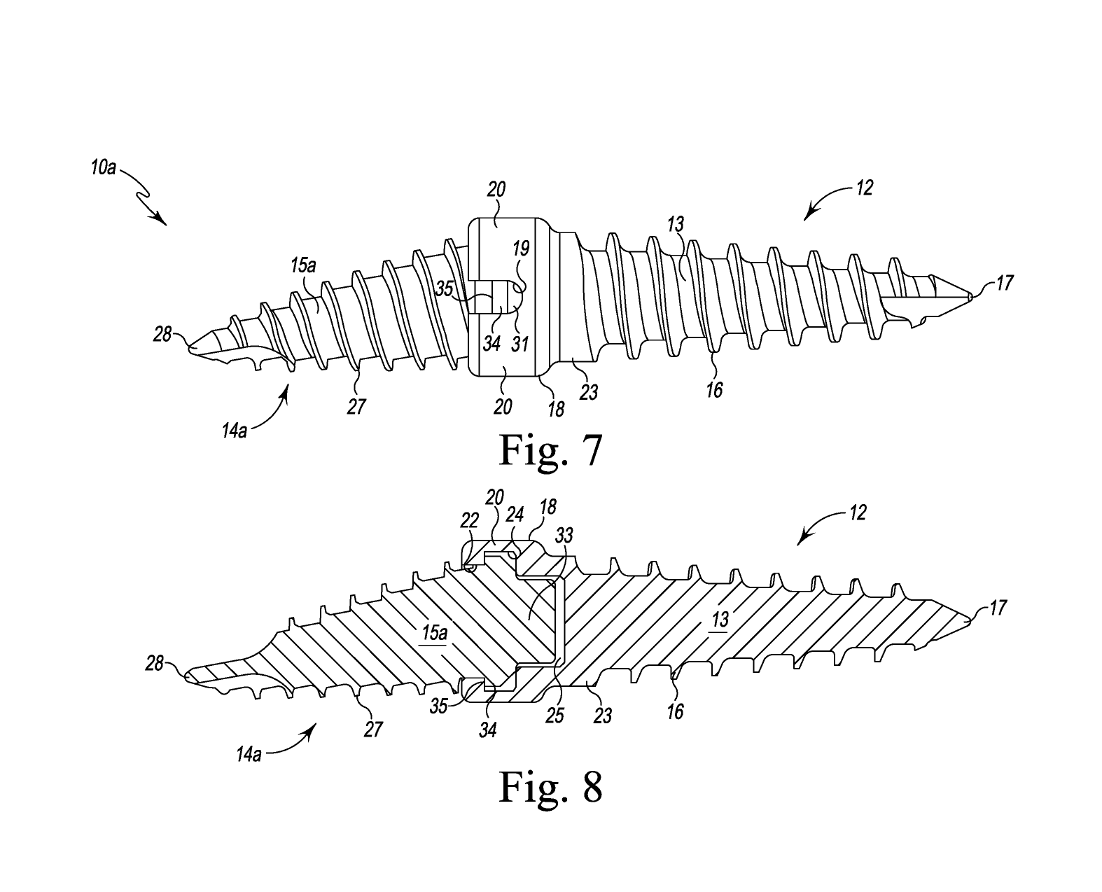

A variation of the small bone orthopedic implant 10 is shown in FIGS. 7-8. Rather than the straight version 10 as described above with reference to FIGS. 1-6, an angled or skewed version 10a is shown with reference to FIGS. 7-8. The small bone orthopedic implant 10a is nearly identical to the small bone orthopedic implant 10 with the exception of those features designated with an “a” next to the callout number. In the figures, the body 15a of the second component 14a is shown as skewed or angled 10° from being co-axial with the first component 12. Other angles may be used. The degree of angle may be dependent upon a desired outcome and/or in view of patient anatomy.

The manner of implanting the small bone orthopedic implant 10a is generally the same as the manner of implanting the small bone orthopedic implant 10. In FIG. 10, the small bone orthopedic implant 10a is shown implanted in the third toe of a left foot LFB while the small bone orthopedic implant 10 is shown implanted in the second toe and the fourth toe of the left foot LFB.

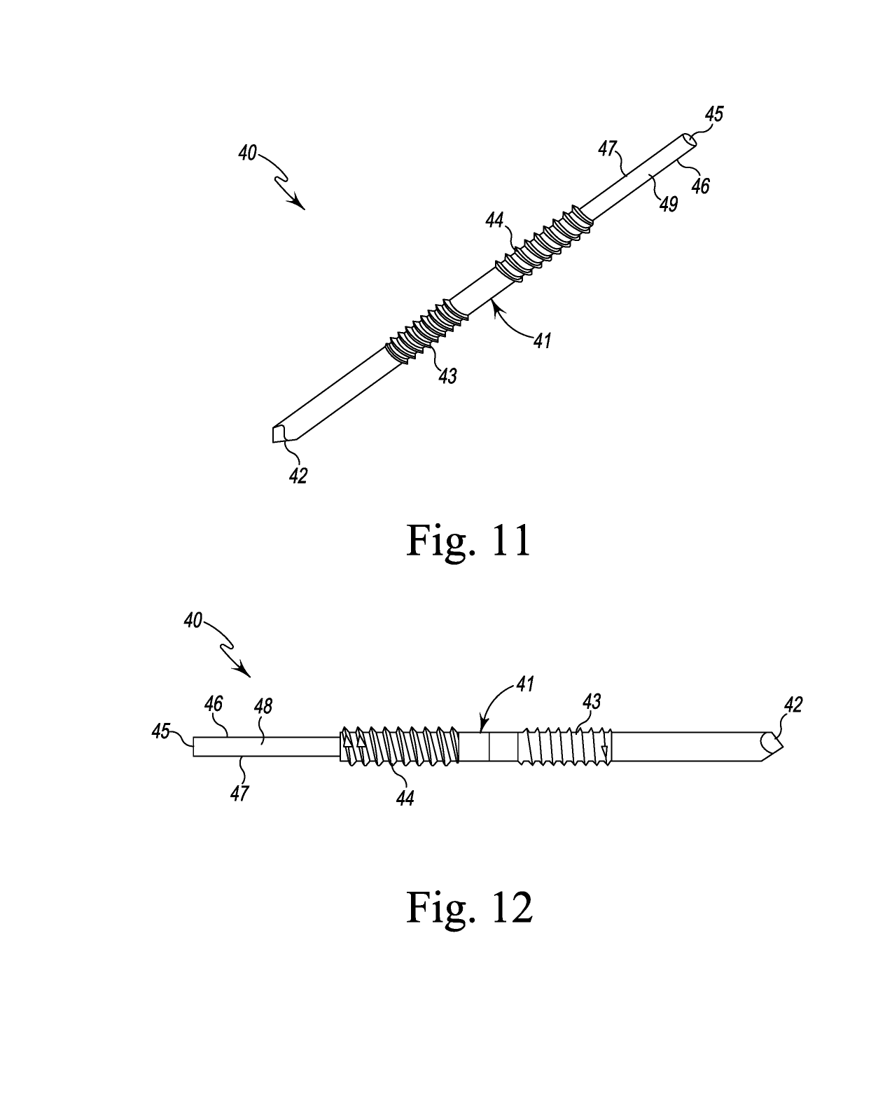

FIGS. 11-16 depict a one component version of the present small bone orthopedic implant, generally designated 40. Like the small bone orthopedic implants 10, 10a, the small bone orthopedic implant 40 is designed for small bone fusion and fractures. The small bone orthopedic implant 40 is characterized by a generally elongated cylindrical body 41 with a distal trocar tip 42 and a proximal driver end 45. The proximal driver end 45 is configured with a first flat 46 and a second flat 47 disposed opposite the first flat 46, the nomenclature first and second being arbitrary. A first rounded end 48 is situated on one side between the first and second flats 46, 47 and a second rounded end 49 is situated on another side between the first and second flats 46, 47. The body 41 further includes distal threads or threading 43 of a first pitch on the outer surface of the body 41 and proximal threads or threading 44 of a second pitch on the outer surface of the body 41. The pitch of the distal threading 43 is different than the pitch of the proximal threading 44.

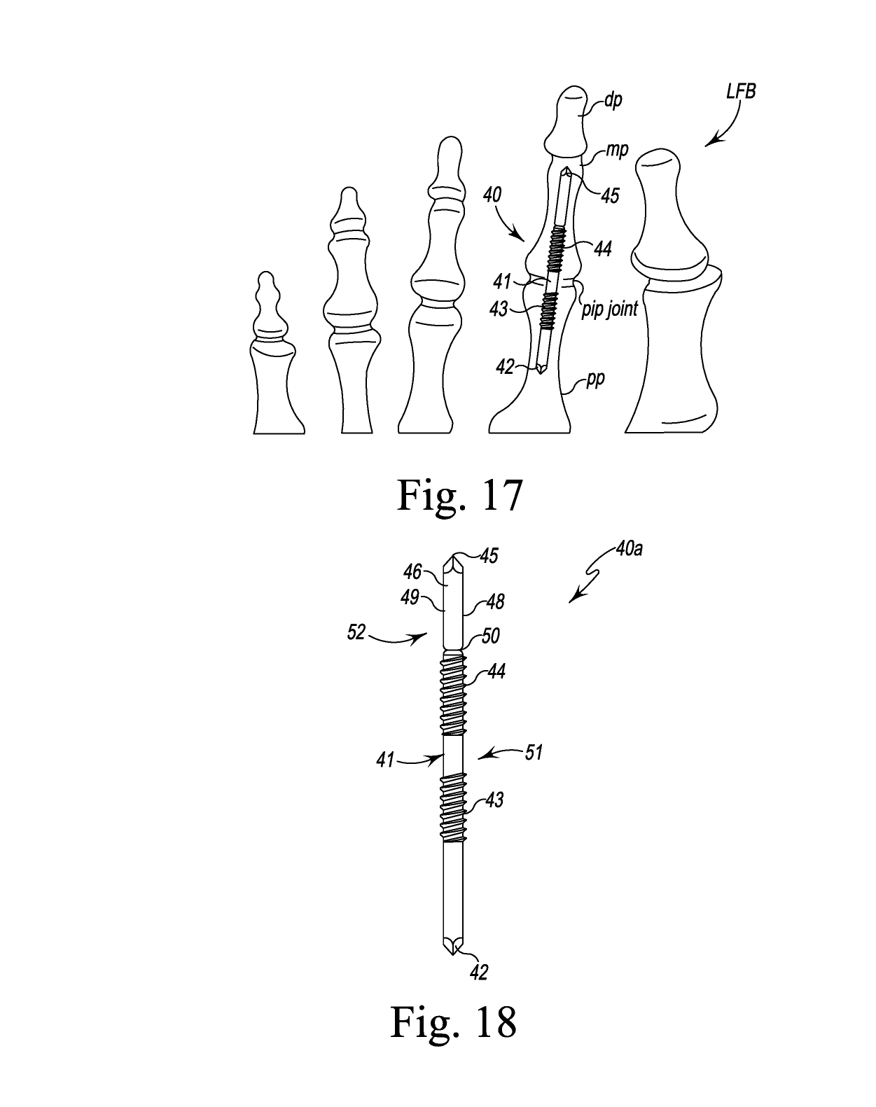

In FIG. 17 the small bone orthopedic implant 40 is shown implanted in a second toe of the left foot LFB between the proximal interphalangeal PIP joint between the proximal phalange PP and the middle phalange MP next to the distal phalange DP. The small bone orthopedic implant 40 is installed in a similar fashion as a K-wire would be installed. After the PIP joint is prepared, the distal trocar tip 42 is driven into the proximal aspect of the middle phalange MP using a driver (not shown) that mates with the proximal driver end 45, and through the distal phalange DP, the implant 40 driven using a clockwise motion. A pilot hole is then made in the distal aspect of the proximal phalange PP. The implant 40 is then driven clockwise (grabbing onto the trocar tip 42) proximally through the distal phalange DP and proximal phalange PP so the proximal threads 44 engage with the pilot hole of the proximal phalange PP. The distal threads 43 and the proximal threads 44 have different pitch so when the proximal threads 44 engage, it causes the joint to compress until the space between the threads occupies the joint space.

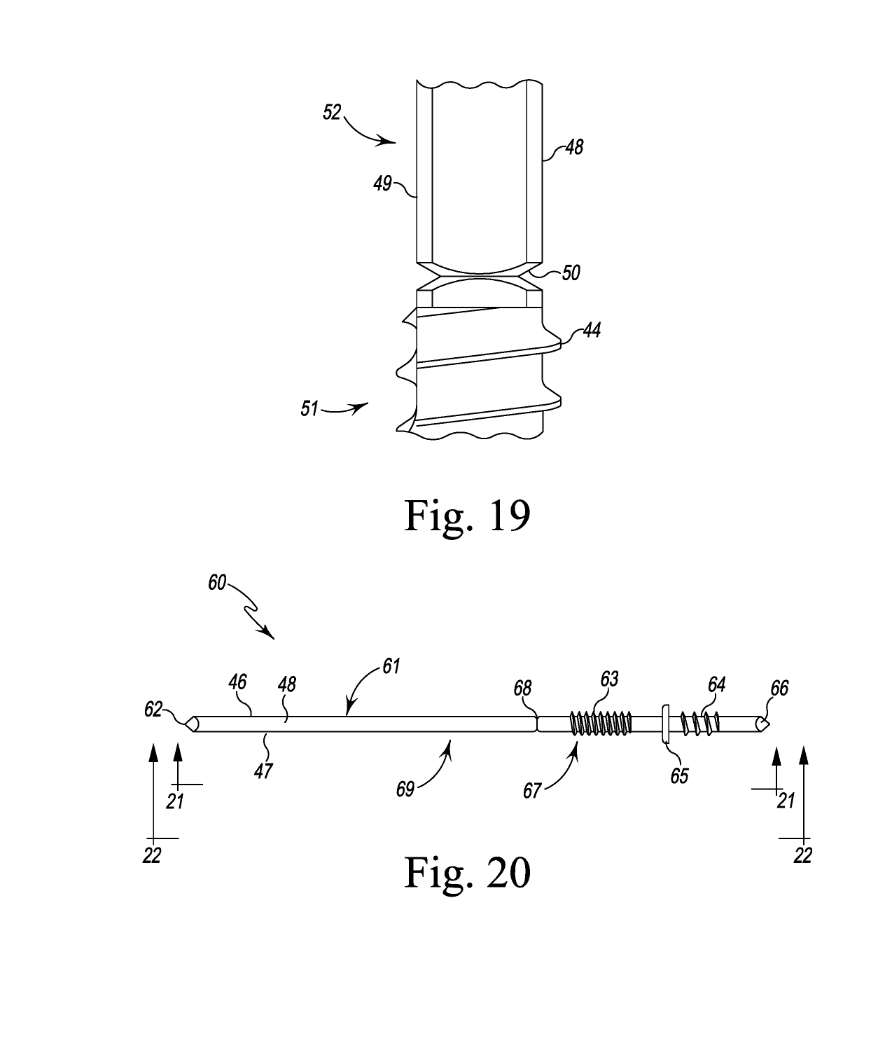

FIGS. 18 and 19 illustrate a variation of the one piece small bone orthopedic implant 40, generally designated 40a. The small bone orthopedic implant 40a has the same features as the small bone orthopedic implant 40 with the exception of a breakaway portion/point 50 where the drive feature is. This allows a driver portion 52 of the implant 40a to be broken off or separated from a screw portion 51 of the implant 40a. Other features are the same.

FIGS. 20-23 depict another one component small bone orthopedic implant 60 with a breakaway feature. Like all of the present small bone orthopedic implants, the small bone orthopedic implant 60 is designed for small bone fusion and fractures. The small bone orthopedic implant 60 is characterized by a generally elongated cylindrical body 61 with a distal trocar tip 66 and a proximal driver end 62. The body 61 further includes distal threads or threading 63 of a first pitch on the outer surface of the body 61 and proximal threads or threading 64 of a second pitch on the outer surface of the body 61. The pitch of the distal threading 63 is different than the pitch of the proximal threading 64. The body 61 also has a breakaway feature 68 that allows a driver portion 69 of the body 61 to be broken off or separated from an implant portion 67 of the body 61. Additionally, a shoulder 65 is provided to the proximal threading 64 that aides in breaking off the distal portion of the implant once installed.

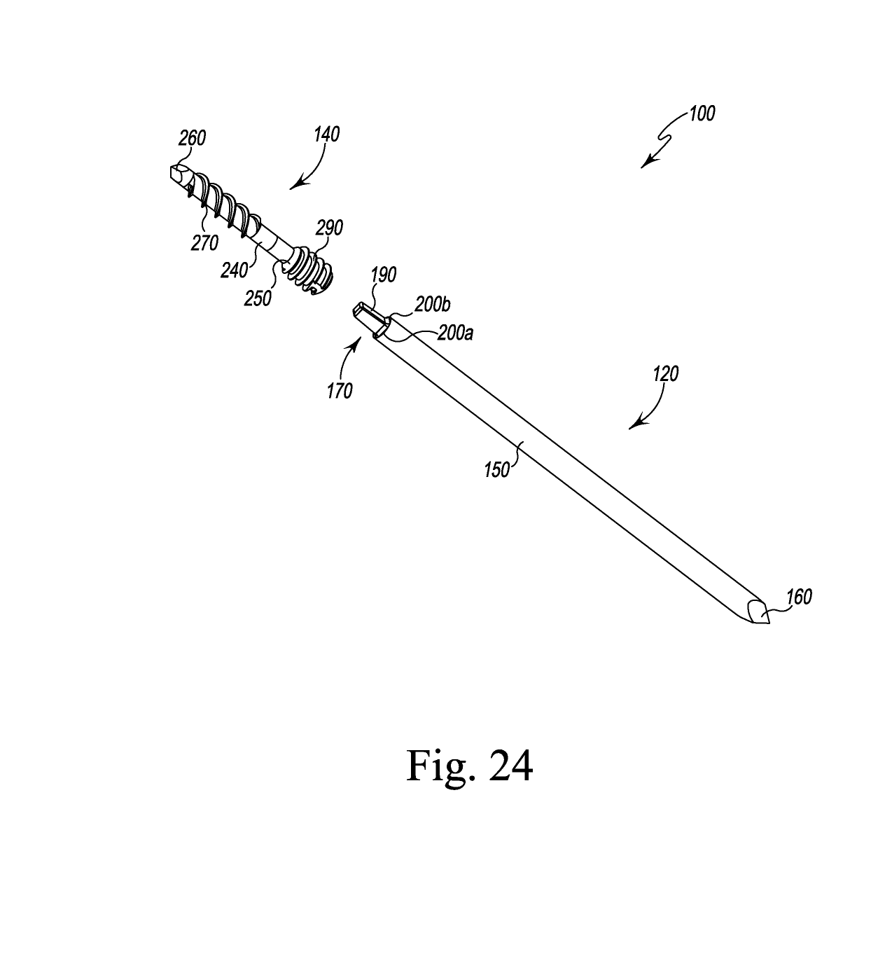

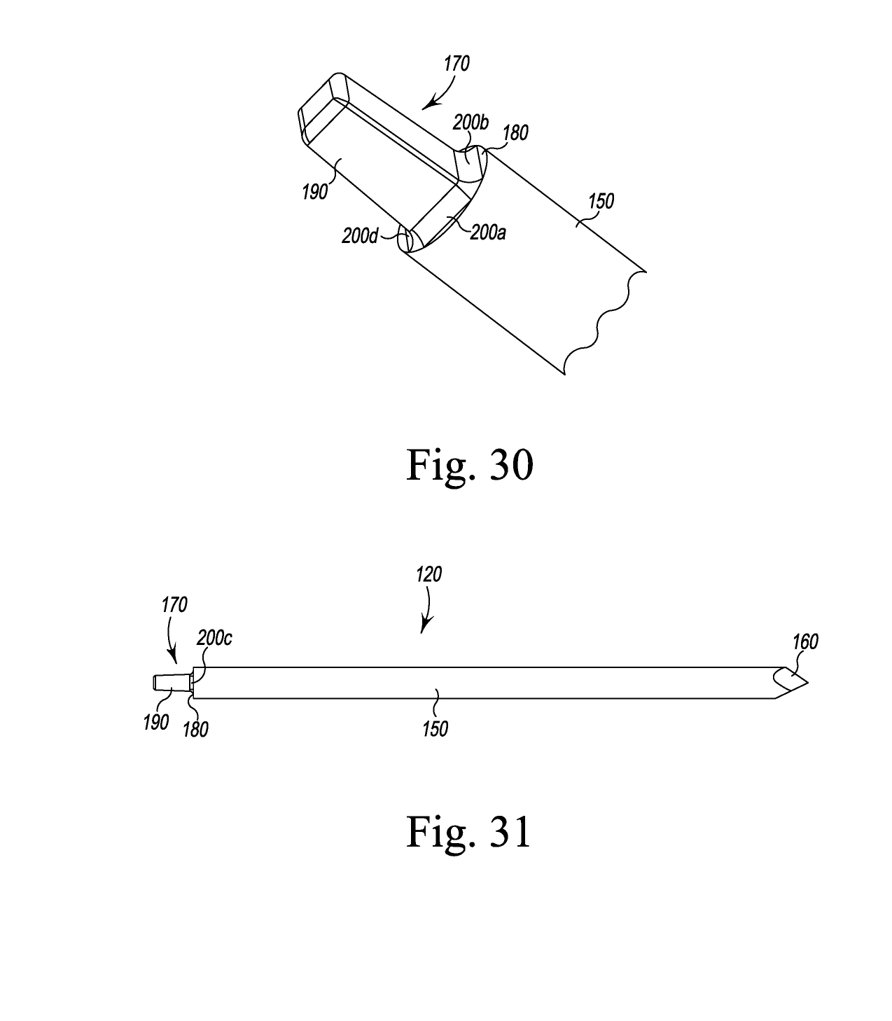

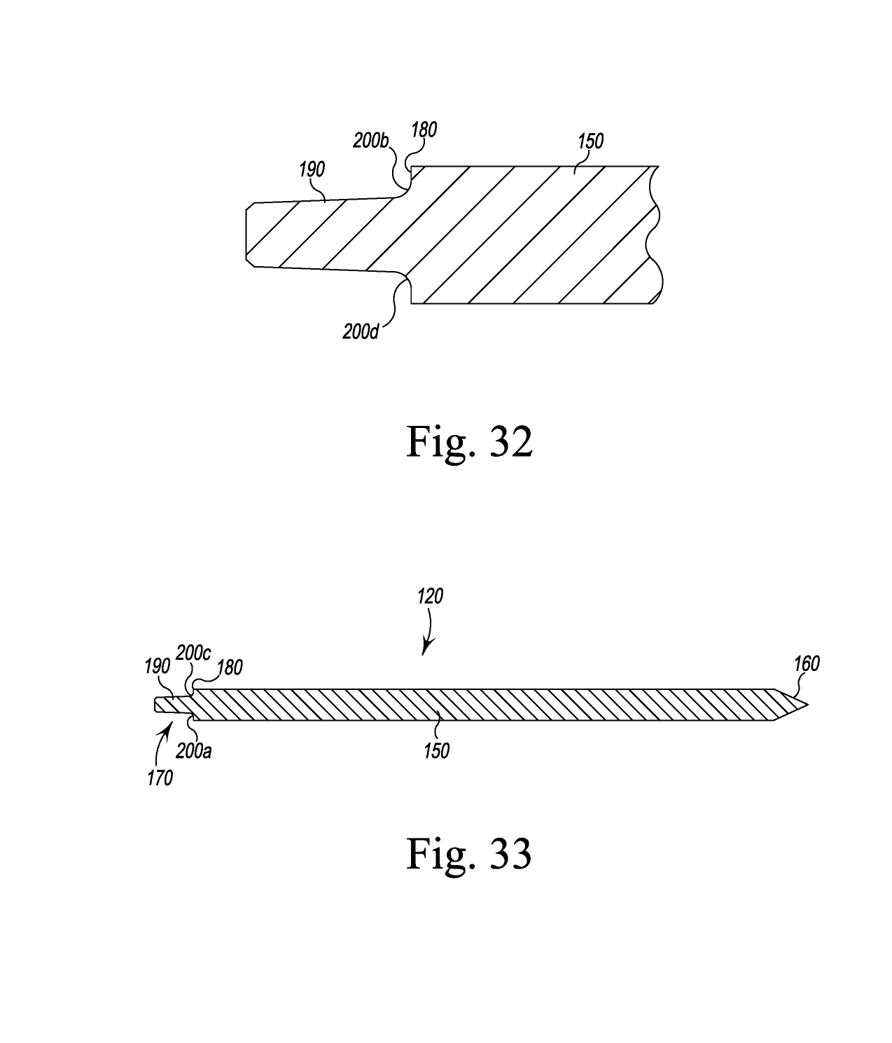

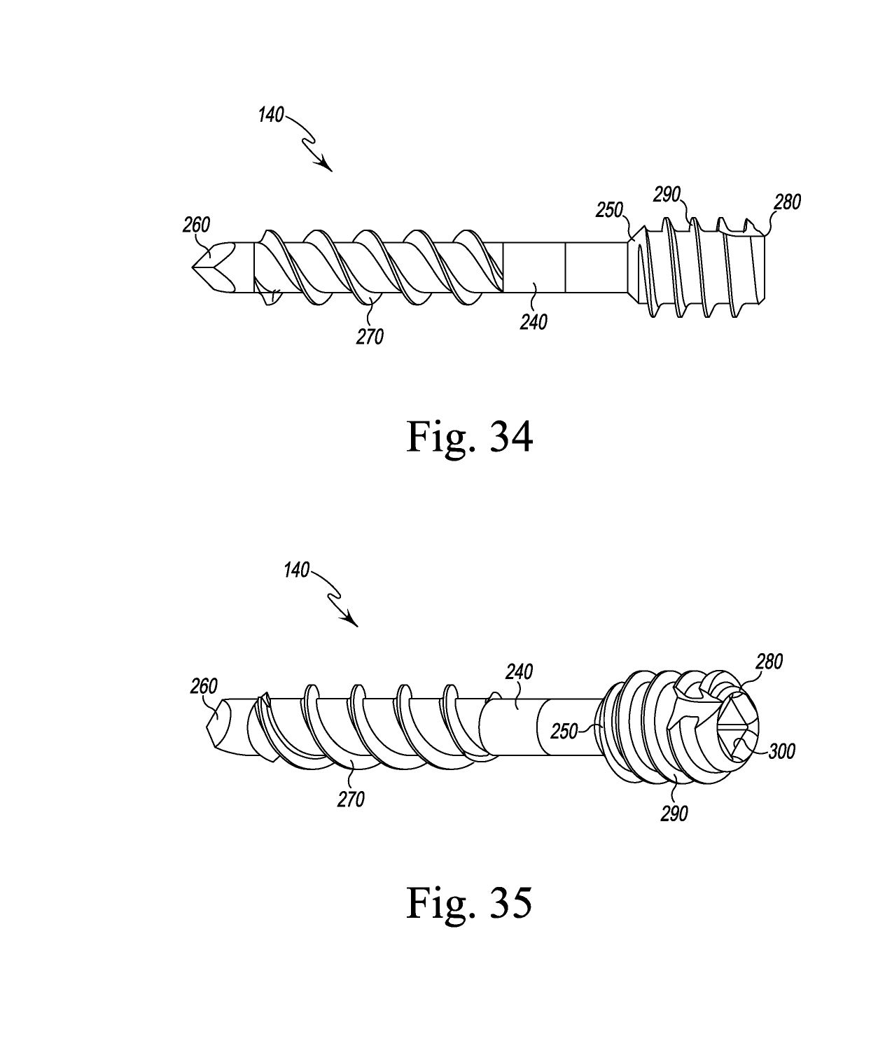

FIGS. 24-38 depict various views of another form or version of the present small bone orthopedic implant fashioned in accordance with the present principles. Like all of the present small bone orthopedic implants, the small bone orthopedic implant 100 of FIGS. 24-38 is designed for small bone fusion and fractures.

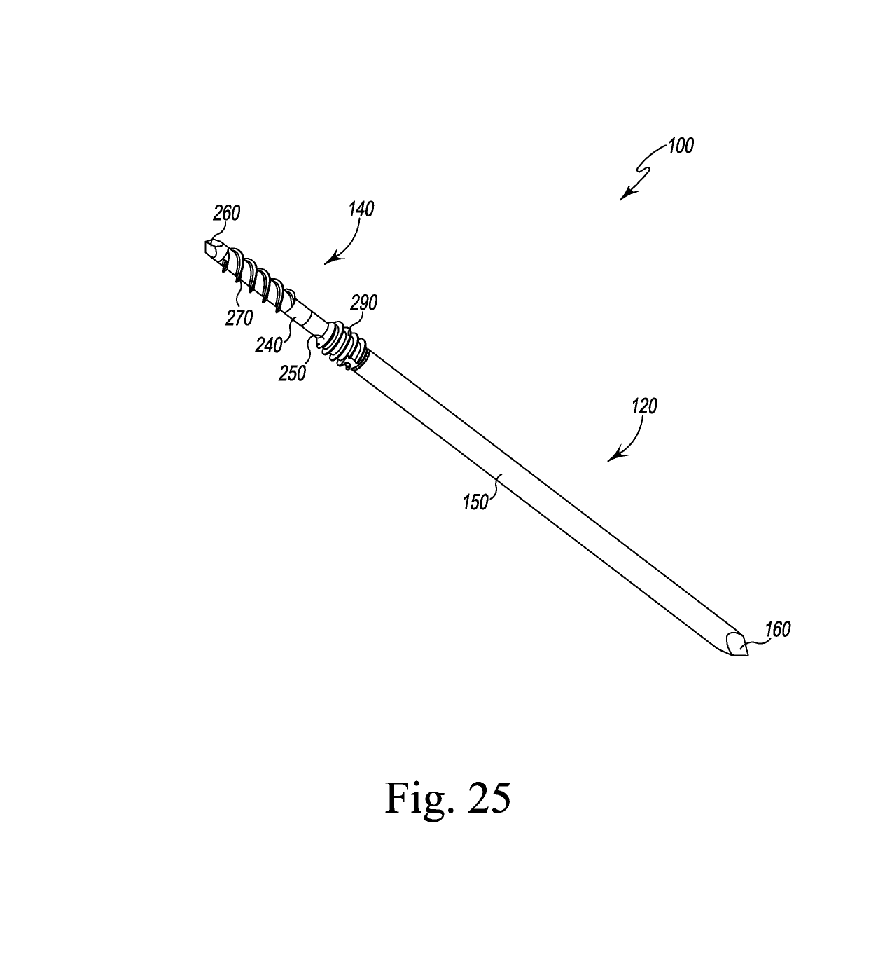

FIG. 24 shows an exploded view of a small bone orthopedic implant assembly generally designated 100 comprising a small bone orthopedic implant (implant) 140 and an implant driver like k-wire (implant driver) 120. The implant driver 120 and the implant 140 are both fashioned from a biocompatible material such as, but not limited to, titanium, a titanium alloy, stainless steel, stainless steel allow, PEEK, or other suitable material. FIG. 25 shows the implant driver 120 engaged with the implant 140 such as when the implant is being installed or implanted.

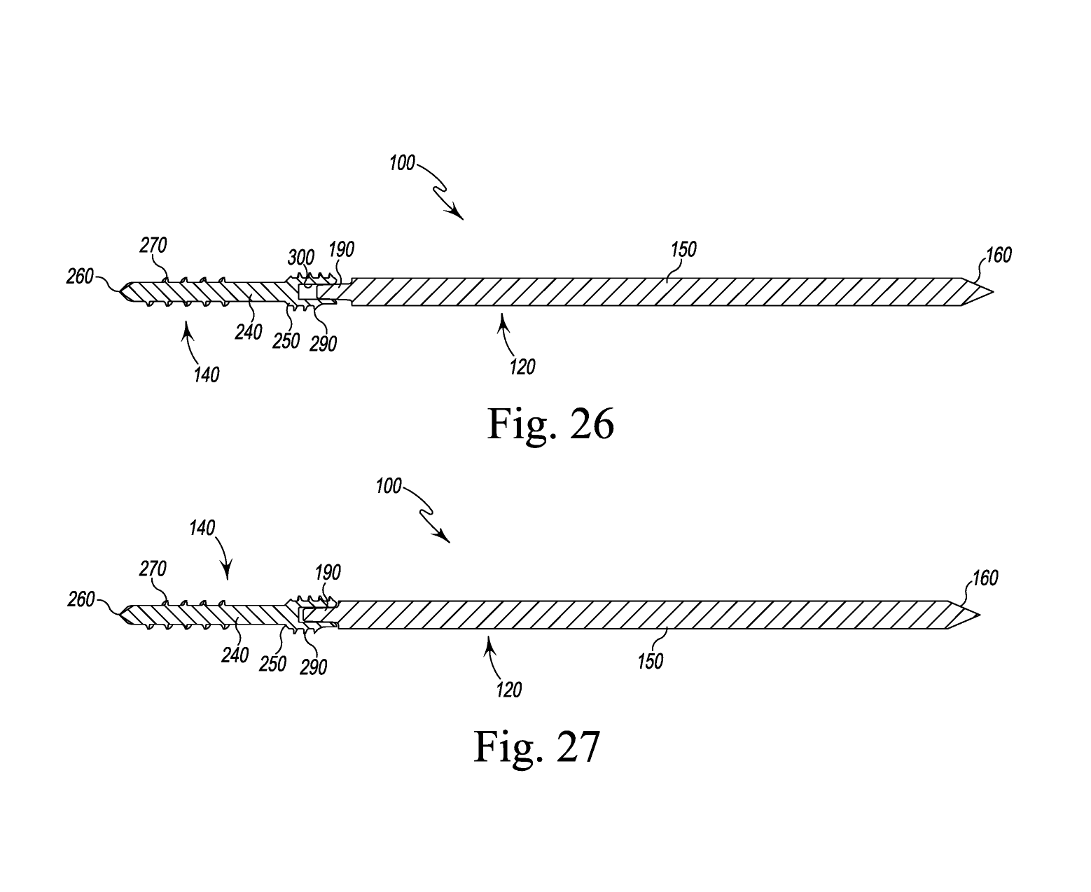

FIGS. 26-37 provide further views of the present invention. The implant driver 120 is characterized by a generally elongated cylindrical rod 150 having a distal tip 160 and a driver 170 on a proximal end 180. The tip 160 is preferably sharp and has a pointed configuration. The driver 170 includes a drive head 190 extending axially from the proximal end 180. While the drive head 190 is shown as square but may be generally rectangular, hexagonal, octagonal, or the like. First, second, third and fourth buttresses 200a, 200b, 200c, 200d (the nomenclature “first”, “second”, “third”, and “fourth” being arbitrary) may be provided (and are shown) at the base of each side the drive head 190 and abutting the proximal end 180. The buttresses 200a, 200b, 200c, 200d provide stability and strength to the drive head 190 during installation/use.

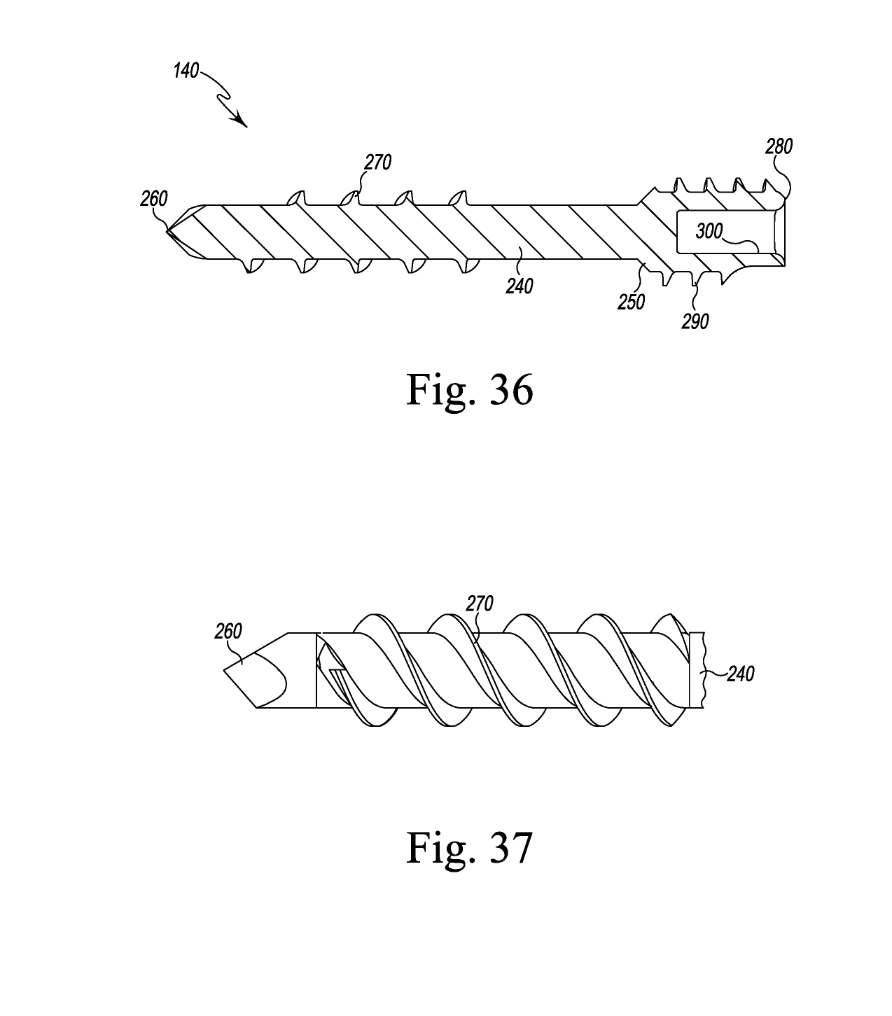

The implant 140 is characterized by a generally cylindrical shaft 240 having a distal tip 260 and a generally planar proximal end 280. A drive socket 300 is provided in the proximal end 280 that is configured in like manner as the drive head 190 of the implant driver 120. The drive socket 300 is therefore square in shape. However, since the drive head 190 may take other shapes as indicated above, the drive socket 300 may likewise take other shapes as desired such as, but not limited to, generally rectangular, hexagonal, octagonal, or the like. As best seen in FIG. 36, the drive socket 300 has a depth at least commensurate with and preferably, but not necessarily, greater than, the length of the drive head 190. This allows the drive head 190 to be fully received within the drive socket 300 (see, e.g., FIGS. 26-29).

The shaft 240 has a proximal thread or threading 270 of a first pitch on the outer proximal surface of the shaft 240 adjacent the tip 260. The shaft 240 has a first shaft diameter making the proximal thread 270 having a first thread diameter. A section 250 at the distal end of the shaft 240 has a second shaft diameter that is larger than the first shaft diameter. The distal section 250 has a distal thread or threading 290 of a second pitch on the outer surface of the distal section 250 having a second thread diameter. The second thread diameter is greater than the first thread diameter. The pitch of the proximal thread 270 is different than the pitch of the distal thread 290.

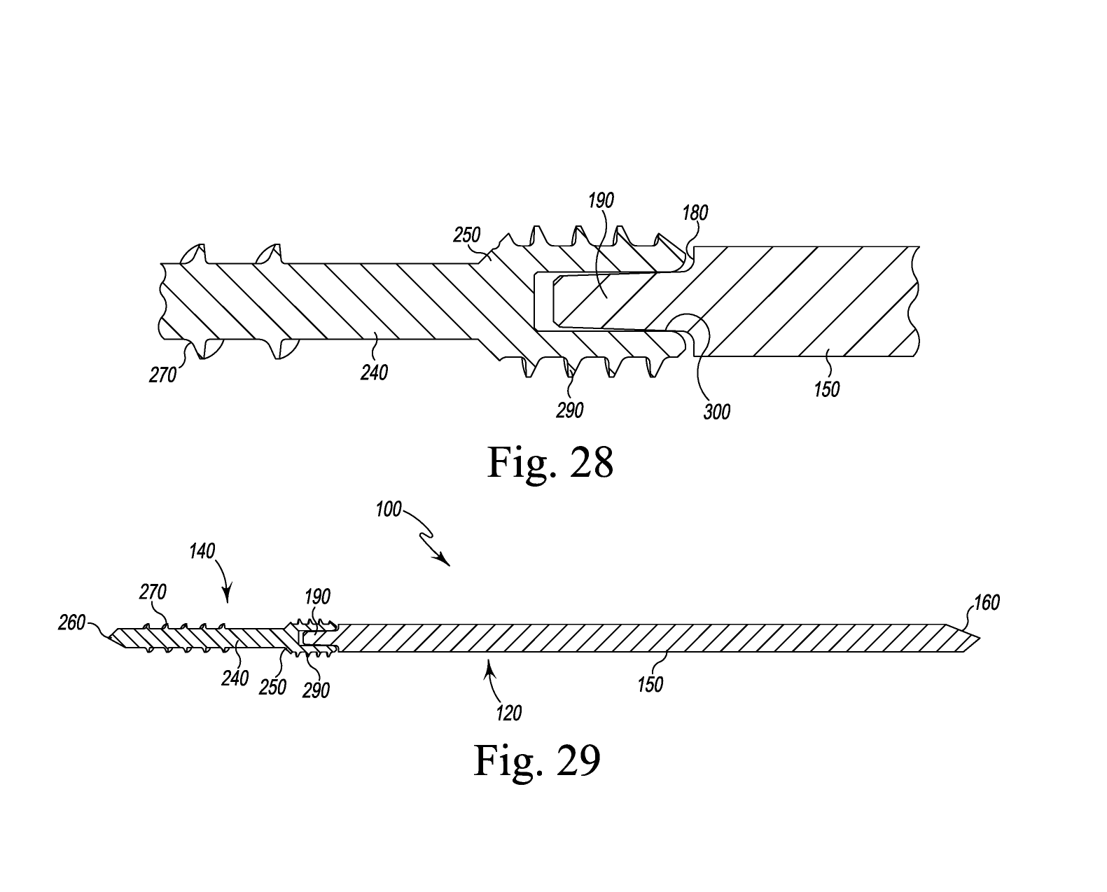

FIG. 26 is a sectional view of the implant driver 120 and the implant 140 with the implant driver 120 beginning to engage the implant 140. Particularly, the drive head 190 of the implant driver 120 is being received in the drive socket 300 of the implant 140. FIGS. 27 and 29 are sectional views of the implant driver 120 fully engaged with the implant 140. Particularly, the drive head 190 of the implant driver 120 has been fully received in the drive socket 300 of the implant 140. FIG. 28 is an enlarged sectional view of end sections of the implant driver 120 showing the drive head 190 and the distal section 250 of the shaft 240 showing the full engagement of the drive head 190 in the drive socket 300.

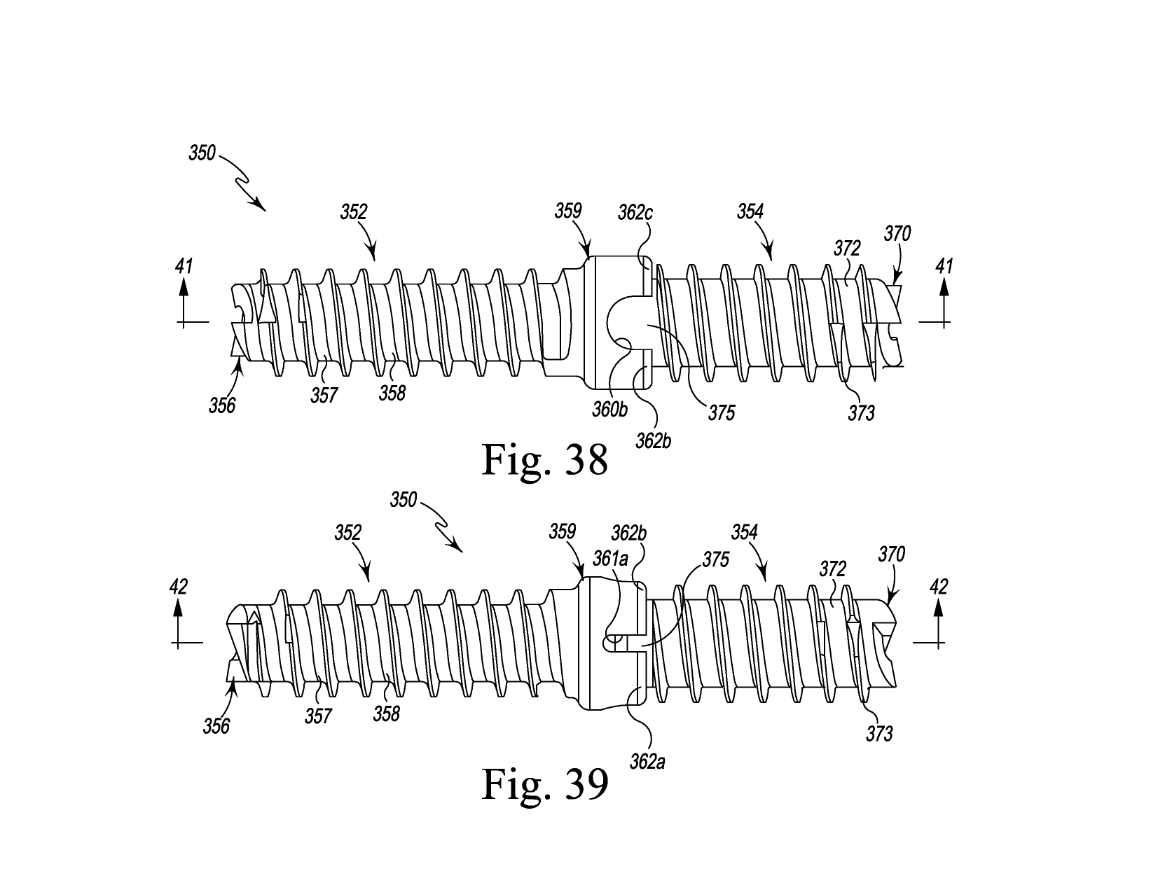

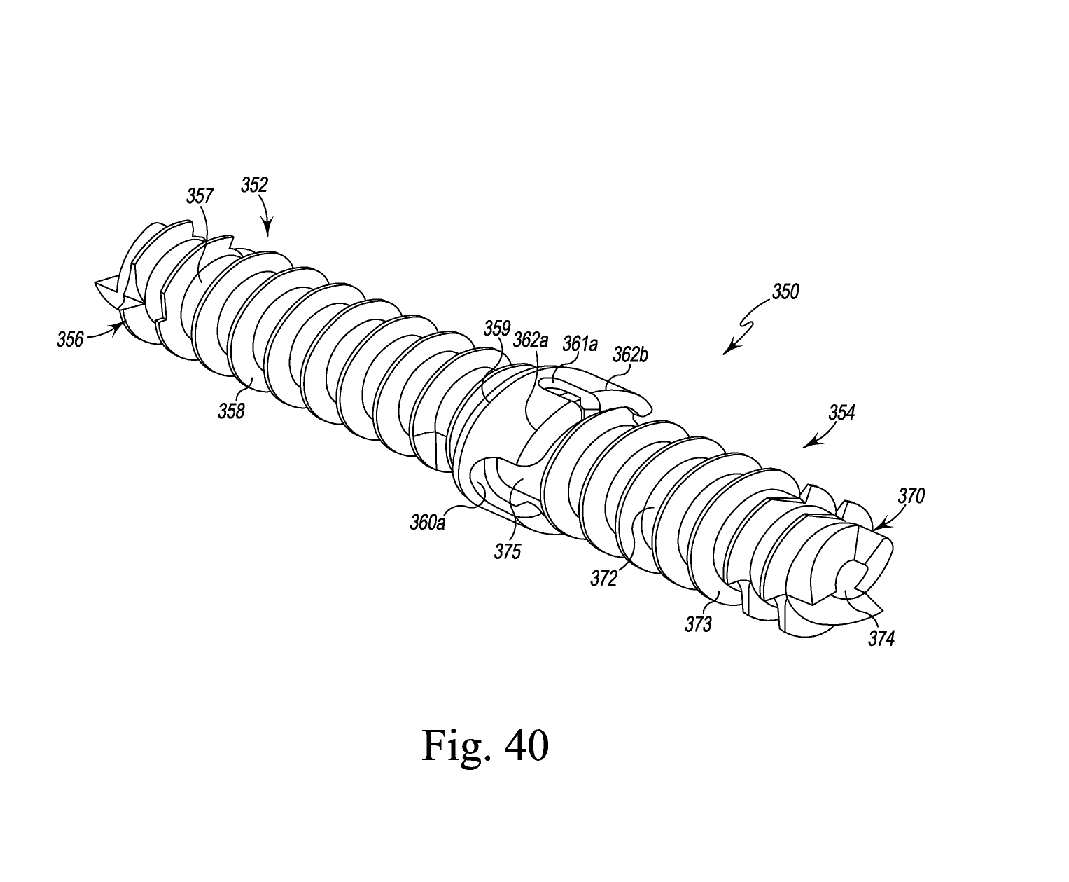

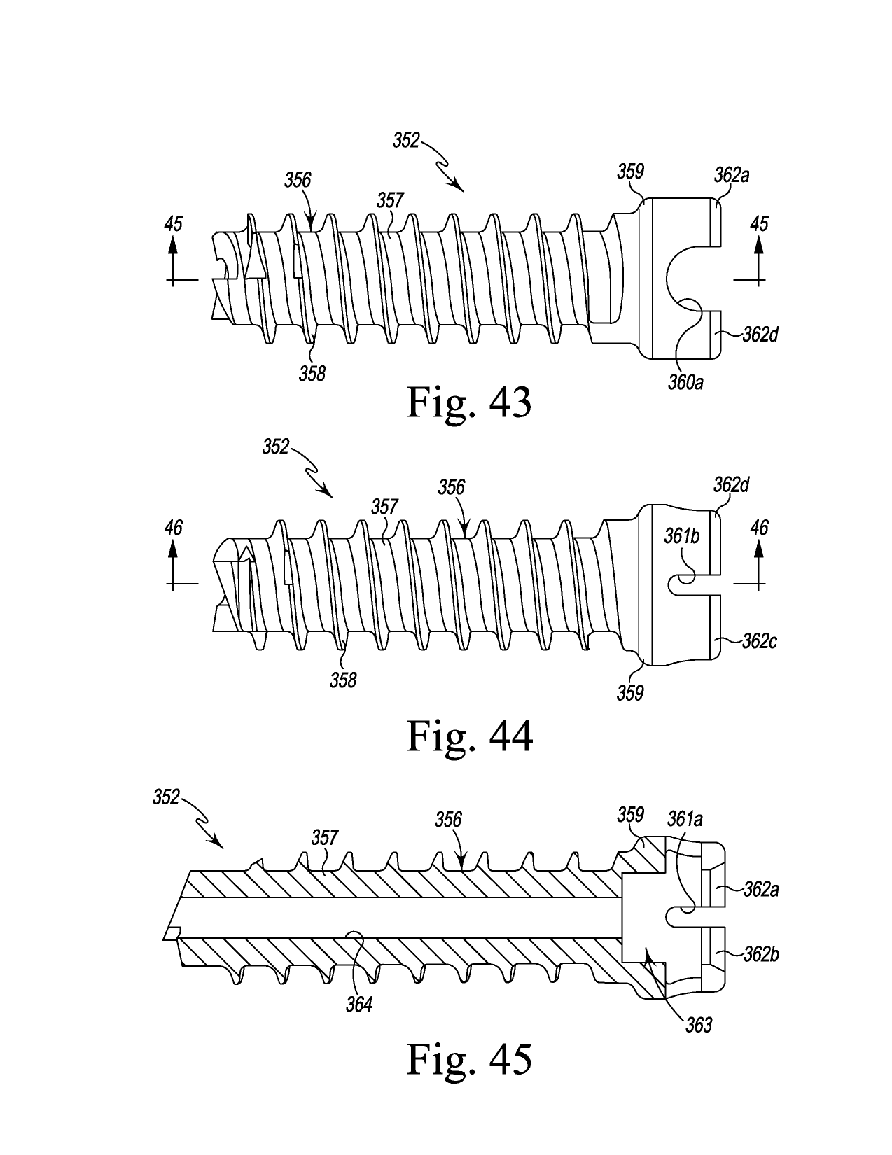

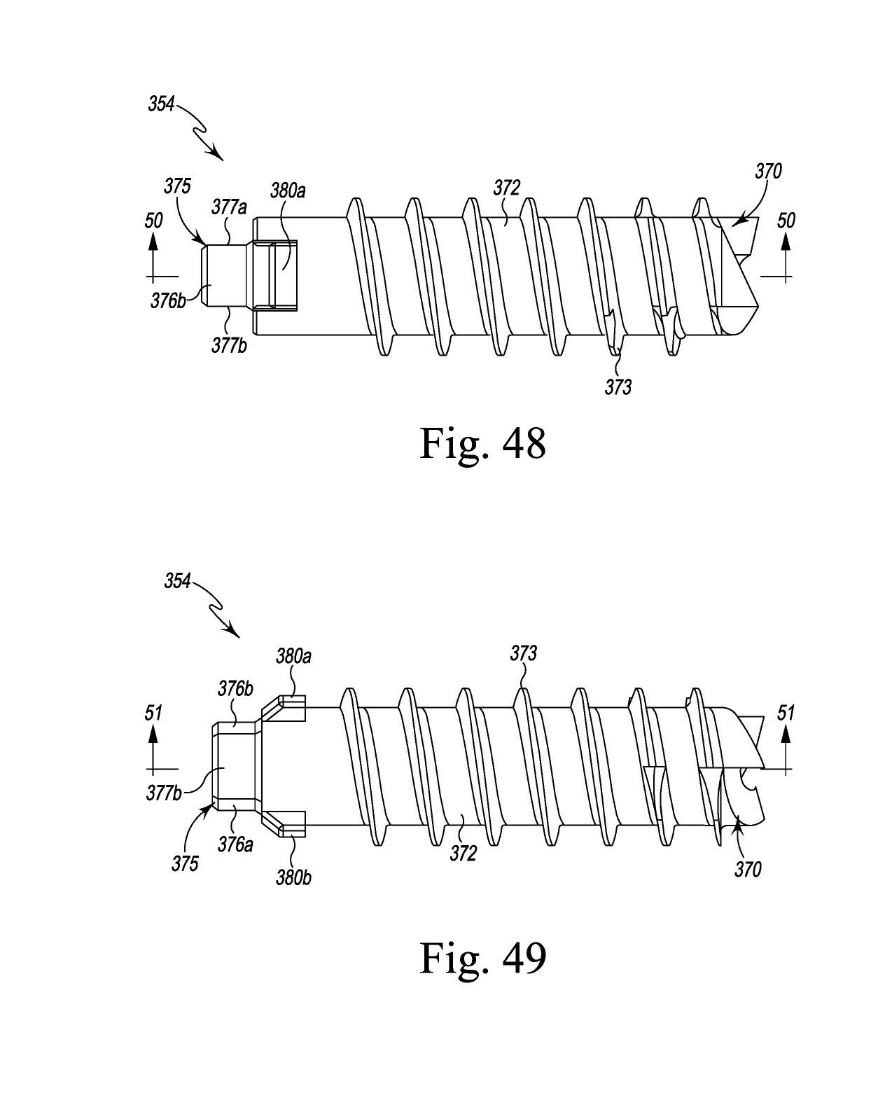

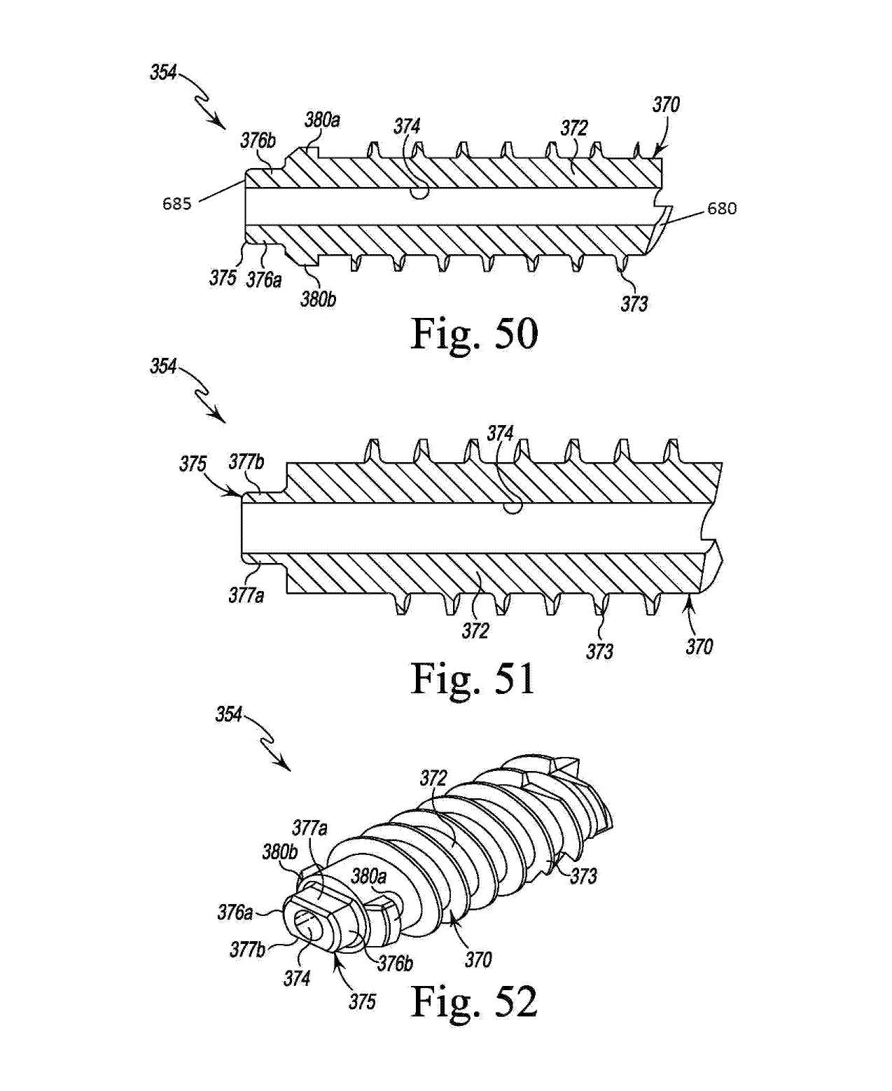

FIGS. 38-52 depict another two component small bone orthopedic implant 350. Like all of the present small bone orthopedic implants, the small bone orthopedic implant 350 is designed for small bone fusion and fractures. The two component small bone orthopedic implant 350 is thus comprised of a first component or segment 352 and a second component or segment 354, the nomenclature first and second being arbitrary. The first and second components 352, 354 are fashioned from a biocompatible material such as, but not limited to, a titanium alloy and are configured to be joined to one another in the implant process. The first component 352 may be considered a female component while the second component 354 may be considered a male component.

The first component 352 is characterized by a body 356 having a generally cylindrical or rod shaped shank or shaft 357, a head 359 at one end of the shaft 357, and a cutting end 659 at another end of the shaft 357 opposite the head 359. The shaft 357 has external threads or threading 358 extending generally from the head 359 to the tip 659. The external threading 358 is sized, shaped and pitched to provide adequate reception and retention in bone.

The head 359 is cup-shaped having a generally rounded wall generally divided into four sections or segments 362a, 362b, 362c, 362d defined by four slots, slits channels or the like 360a, 360b, 361a, 361b formed in the walls 362a, 362b, 362c, 362d. The walls 362a, 362b, 362c, 362d at least in part define a socket 363. The slots 360a and 360b are preferably, but not necessarily, wider than the slots 361a and 361b. Moreover, the slots 360a, 360b are situated opposite one another, while the slots 361a, 361b are likewise situated opposite one another. Other slot size, placement and configurations are contemplated. The socket 363 is configured in like manner as the boss 375 of the second component 354 (which is described more fully below). While the socket 363 and thus the boss 375 is shown as generally rectangular in shape, the socket 363 and the boss 375 may be configured in other shapes as desired. The head 359 is thus configured to allow reception of a tool (not shown) to drive the first component 352 into a small bone of the body.

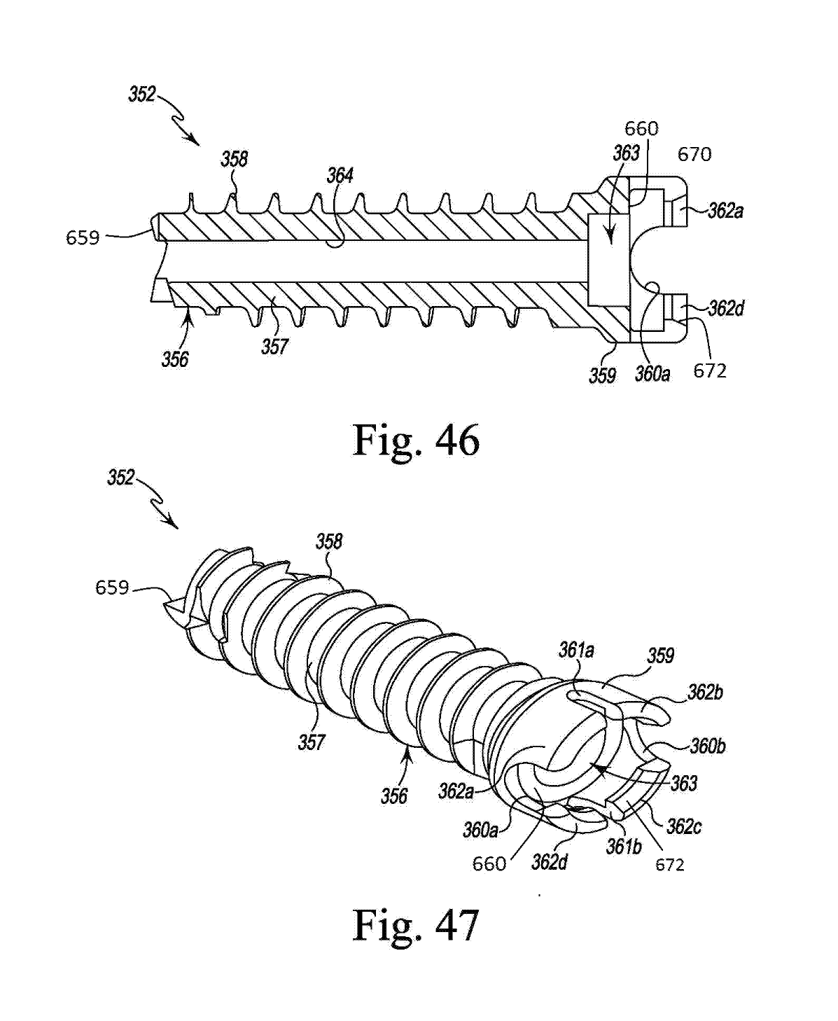

As seen in FIGS. 46 and 47, but not necessarily limited thereto, the inside of the walls 362a, 362b, 362c, 362d have an undercut or ledge 670 while the outside of the walls 362a, 362b, 362c, 362d are angled 672. The undercut 670 defines a generally annular retention tang providing a mating feature for coupling with the second component 354. The angle 672 provides guided reception or insertion of the second component 354 into the socket 363. The slots 362a, 362b, 362c, 362d in the walls 362a, 362b, 362c, 362d allow for the walls to expand slightly when the second component 354 is inserted into the head 359 but resiliently return to their original shape after insertion of the second component 354. The inside of the socket provides communication with a central bore, cannula or the like 364 that extends the longitudinal length of the body 356. A ledge 660 is formed about the bore 364.

The second component 354 is characterized by a generally cylindrical shaped body 370 having a shaft or shank 372, a head or boss 375 at one end thereof, and a general cutting tip or end 680 at another end thereof. The shaft 372 has external (e.g. distal cortical) threads or threading 373 extending generally from near the head 375 to the tip 680. The external threading 373 is sized, shaped and pitched to provide adequate reception and retention in bone.

The head or boss 375 is generally rectangular in shape having two generally planar and opposite sides 377a, 377b, but having two, opposite ends 376a, 376b that are generally outwardly rounded or arched, and terminating in a generally flat top 685. The boss or drive feature 375 is situated on and extends axially from the end of the shaft 372. The boss 375 is configured for reception in the socket 363 of the first component 352. The boss 375 may take other shapes as desired.

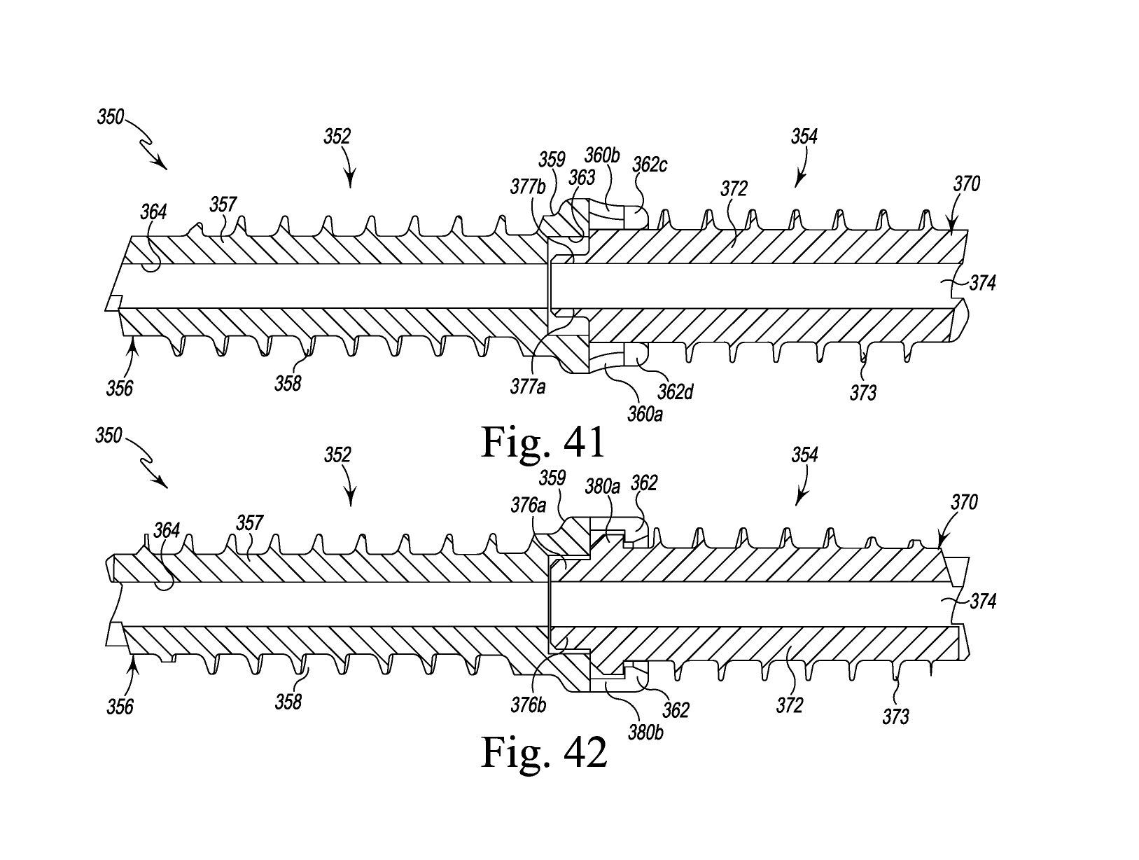

As seen at least in FIGS. 49, 50, and 52, the area of the head 375 of the second component 354 includes a proximal mating feature 380 that retains the second component 354 onto the first component 352 preventing disassociation between the two components. In particular, the body 370 has first and second tangs 380a, 380b situated axially between the boss 375 and the external threading 373. The two tangs 380a, 380b are preferably, but not necessarily, disposed opposite one another. Moreover, the tangs 380a, 380b are preferably, but not necessarily, disposed axially adjacent respective rounded ends 376a, 376b of the boss 375. As seen in at least FIG. 42, the tangs 380a, 380b are received within the socket 363 of the head 359 of the first component 352 and, particularly, underneath the undercut 670 of the head walls 362a, 362b, 362c, 362d. Other configurations may be used.

The two component small bone orthopedic implant 350 is designed such that the longitudinal axis of the first component 352 and the longitudinal axis of the second component 354 are co-axial or straight relative to one another. However, the longitudinal axes of the two components may be skewed or angled relative to one another if desired.

It should be appreciated that dimensions of the components, structures, and/or features of the present small bone orthopedic implants can be altered as desired.

What is claimed is:

1. An orthopedic implant for treating small bones of the hand and foot comprising:
a component having a first segment and a second segment;
first external threading on the first segment of the component; and
second external threading on the second segment of the component.

2. The orthopedic implant of claim 1, wherein:
the first external threading provides rotation in a first direction;
the second external threading provides rotation in a second direction; and
the first and second directions are opposite one another.

3. The orthopedic implant of claim 1, wherein the first segment and the second segment are one member.

4. The orthopedic implant of claim 3, wherein the first segment and the second segment are separated by a break-away portion that allows the first segment to be separated from the second segment.

5. The orthopedic implant of claim 1, wherein the first segment and the second segment are separate first and second members.

6. The orthopedic implant of claim 5, wherein:
the first member has a first end and a second end, the first end of the first member having a head; and
the second member has a first end and a second end, the first end of the second member having a boss configured for joining reception with the head of the first end of the first member.

7. The orthopedic implant of claim 6, wherein:
the second end of the first member has a first generally planar bone cutting configuration; and
the second end of the second member has a second generally planar bone cutting configuration.

8. The orthopedic implant of claim 6, wherein:
the second end of the first member has a first generally pointed bone cutting configuration; and
the second end of the second member has a second generally pointed bone cutting configuration.

9. The orthopedic implant of claim 6, wherein the head of the first end of the first member has a socket configured to receive the boss of the first end of the second member.

10. The orthopedic implant of claim 9, wherein a wall defining the head of the first end of the first member includes an inner radial ledge configured to receive a connection structure on the first end of the second member.

11. The orthopedic implant of claim 10, wherein the connection structure on the first end of the second member comprises radially extending tangs.

12. The orthopedic implant of claim 10, wherein the wall has a plurality of vertical slots.

13. An orthopedic implant for use in treating small bones of the body comprising:
a component having a shaft with a first end and a second end;
first threading proximate the first end of the shaft;
second threading proximate the second end of the shaft;
a tip on the first end of the shaft; and
a driver socket on the second end configured to receive a driver.

14. The orthopedic implant of claim 13, wherein the second end of the shaft has a diameter that is greater than the first end of the shaft.

15. An orthopedic implant for treating small bones of the hand and foot comprising:
a first component defining a first end and a second end;
a head disposed at the first end of the first component;
a second component defining a first end and a second end, the first end of the second member having a boss configured for joining reception with the head of the first end of the first component;
first external threading on the first component, the first external threading providing insertion rotation of a first direction; and
second external threading on the second component, the second external threading providing insertion rotation of a second direction, the first and second insertion directions opposite one another.

16. The orthopedic implant of claim 15, wherein:
the second end of the first component has a first generally planar bone cutting configuration; and
the second end of the second component has a second generally planar bone cutting configuration.

17. The orthopedic implant of claim 15, wherein:
the second end of the first component has a first generally pointed bone cutting configuration; and
the second end of the second component has a second generally pointed bone cutting configuration.

18. The orthopedic implant of claim 17, wherein:
the head of the first end of the first component has a socket configured to receive the boss of the first end of the second member; and
a wall defining the head of the first end of the first component includes an inner radial ledge configured to receive a connection structure on the first end of the second component, the wall having a plurality of vertical slots.

19. The orthopedic implant of claim 18, wherein the connection structure on the first end of the second component comprises radially extending tangs.

20. A method of treating small bones of the foot and hand comprising:
surgically isolating bone(s) requiring treatment;
surgically treating the bone(s) for reception of an orthopedic implant; and
implanting an orthopedic implant for treating small bones of the hand and foot comprising:
a first component defining a first end and a second end;
a head disposed at the first end of the first component;
a second component defining a first end and a second end, the first end of the second member having a boss configured for joining reception with the head of the first end of the first component;
first external threading on the first component, the first external threading providing insertion rotation of a first direction; and
second external threading on the second component, the second external threading providing insertion rotation of a second direction, the first and second insertion directions opposite one another.
